(19) United States
(12) Patent Application Publication
(10) Pub. No.: US 2015/0104806 A1
(43) Pub. Date: Apr. 16, 2015

(54) Parkinson's Disease Biomarker

(71) Applicant: University of Dundee, Dundee (GB)

(72) Inventors: Dario Alessi, Dundee (GB); Muratul Muqit, Dundee (GB)

(73) Assignee: University of Dundee, Dundee (GB)

(21) Appl. No.: 14/391,372

(22) PCT Filed: Apr. 10, 2013
(86) PCT No.: PCT/GB2013/050926

§ 371 Date: Oct. 8, 2014

(30) Foreign Application Priority Data
Apr. 11, 2012 (GB) 1206382.2

(51) Int. Cl. G01N 33/573; C07K 16/40

(52) U.S. Cl. CPC G01N 33/573; C07K 16/40; G01N 2800/2835; G01N 2333/91205
USPC 435/7.4; 530/389.8

(57) ABSTRACT

The present invention relates to a biomarker for Parkinson's disease. The biomarker and products associated with the biomarker may be used to assist diagnosis or to assess onset and/or development of Parkinson's disease. The invention also relates to use of the biomarker in clinical screening, assessment of prognosis, evaluation of drug treatments, drug screening or drug development in the field of Parkinson's disease and Parkinson's disease related disorders.

### FIELD OF THE INVENTION

The present invention relates to a biomarker for Parkinson's disease. The biomarker and products associated with the biomarker may be used to assist diagnosis or to assess onset and/or development of Parkinson's disease. The invention also relates to use of the biomarker in clinical screening, assessment of prognosis, evaluation of drug treatments, drug screening or drug development in the field of Parkinson's disease and Parkinson's disease related disorders.

### BACKGROUND TO THE INVENTION

Human PINK1 encodes a 581 residue serine-threonine kinase unique amongst all protein kinases since it contains an N-terminal mitochondrial targeting motif (residues 1 to 34) (Muqit et al, 2006; Valente et al, 2004). The catalytic domain of PINK1 (residues 150 to 513) is not closely related to other protein kinases and is also unusual in that it possesses three unique insertions between the beta strands that make up the typical fold of the N-lobe of protein kinases, (Woodroof et al., 2011). PINK1 contains a conserved C-terminal non-catalytic region of unknown function (residues 514 to 581). Great excitement in understanding the regulation and function of this enzyme resulted from the 2004 landmark discovery that loss of function autosomal-recessive mutations in PINK1 caused early onset Parkinson's disease (Valente et al, 2004). Subsequent studies in flies revealed that Drosophila PINK1 null mutants share many overlapping features with human Parkinson's disease, including motor deficits, neuronal loss and mitochondrial abnormalities (Clark et al, 2006) (Park et al, 2006). Other work in Drosophila (Yang et al, 2008) suggests that PINK1 plays a role in regulating mitochondrial dynamics. For example over-expression of PINK1 enhances mitochondrial fission whilst loss of PINK1 leads to excess fusion (Yang et al, 2008).

Recent work in mammalian cells provides further links between PINK1 and the mitochondria. Current data suggests that following recruitment of PINK1 to the mitochondrial membrane via its N-terminal targeting sequence, it is subsequently proteolysed between residues Ala103-Phe104 by the mitochondrial rhomboid protease, PARL (Deas et al, 2011; Jin et al, 2010; Meissner et al, 2011; Whitworth et al, 2008), resulting in a processed form of PINK1 which is rapidly degraded by the 20S proteasome (Muqit et al, 2006; Takatori et al, 2008). In response to mitochondrial depolarisation, for example induced by the uncoupling agent carbonyl cyanide m-chlorophenyl hydrazone (CCCP), a marked stabilisation of full length PINK1 at the mitochondria is observed (Narendra et al, 2010). How CCCP stabilises full length PINK1 is not known, but one proposal is that mitochondrial depolarisation might result in a loss of function of the PARL protease (Meissner et al, 2011).

Despite considerable research, we still have limited knowledge on the mechanism by which PINK1 kinase activity is regulated and what substrates it might phosphorylate physiologically and how this links to Parkinson's disease. The present inventors and many other groups have observed that recombinant PINK1 expressed in mammalian cells is inactive, which has limited the ability to utilise traditional biochemical approaches to identify PINK1 substrates.

Autosomal-recessive inherited mutations in parkin are one of the most frequent causes of familial Parkinson's disease especially young-onset forms (Kitada et al, 1998). Previous genetic analysis in Drosophila has suggested significant links between PINK1 and parkin (Clark et al, 2006; Park et al, 2006) and human patients with mutations in either of these enzymes display very similar clinical symptoms (Abeliovich & Flint Beal, 2006).

### SUMMARY OF THE INVENTION

The present invention provides biological markers (“biomarkers”) relating to Parkinson's disease. These biomarkers can be used to diagnose the disease, monitor its progression, assess response to therapy and screen drugs for treating Parkinson's disease. Early diagnosis and knowledge of disease progression could allow early treatment when it is most appropriate and would be of the greatest benefit to the patient. In addition, such information will allow prediction of exacerbations and classification of potential Parkinson's disease subtypes. The ability to evaluate response to therapy may allow personalized treatment of the disease and/or support clinical trials aimed at evaluating the effectiveness of candidate drugs.

The biomarkers of the present invention include phosphorylation of amino acid residues of the parkin and PINK1 protein. In particular the present invention includes the identification of phosphorylation of the Ser⁶⁵ residue of parkin (numbering according to GenBank: BAA25751.1) which the inventors have observed as being phosphorylated by PINK1. As well as phosphorylation of the identified Serine residue, the inventors have observed that following activation, PINK1 autophosphorylates at Thr²⁵⁷ and studying this phosphorylation site may also be of relevance. In one embodiment, the invention provides a method for determining whether a subject has Parkinson's disease, by studying phosphorylation of parkin, especially phosphorylation of Ser⁶⁵ and/or phosphorylation of PINK1 at Thr²⁵⁷.

In related embodiments, the invention provides a method for determining whether a subject is more likely than not to have Parkinson's disease, or is more likely to have Parkinson's disease than to have another disease.

The method is performed by analysing a biological sample, such as serum or CSF from the subject; measuring the level of phosphorylation of at least one of the biomarkers in the biological sample; and optionally comparing the measured phosphorylation level with a standard level or reference range. Typically, the standard level or reference range is obtained by measuring the same marker or markers in a normal control or, more preferably, a set of normal controls. Depending upon the difference between the measured level and the standard level or reference range, the patient can be diagnosed as having or being predisposed to developing Parkinson's disease, or as not having Parkinson's disease. As will be appreciated by one of skill in the art, a standard level or reference range is specific to the biological sample at issue. Thus, a standard level or reference range for the marker in serum that is indicative of Parkinson's disease would be expected to be different from the standard level or reference range (if one exists) for that same marker in CSF, urine or another tissue, fluid or compartment. Thus, references herein to measuring biomarkers will be understood to refer to measuring the level of phosphorylation of the biomarker. Furthermore, references herein to comparisons between a marker phosphorylation measurement level and a standard level or reference range will be understood to refer to such levels or ranges for the same type of biological sample.

In another embodiment, the invention provides a method for monitoring a Parkinson's disease patient over time to determine whether the disease is progressing. The method is performed by analysing a biological sample, such as serum or CSF, from the subject at a certain time; measuring the phosphorylation level of at least one of the biomarkers in the biological sample; and comparing the measured phosphorylation level with the phosphorylation level measured with respect to a biological sample obtained from the subject at an earlier time. Depending upon the difference between the measured phosphorylation levels, it can be seen whether the marker phosphorylation level has increased, decreased, or remained constant over the interval. Subsequent sample acquisitions and measurements can be performed as many times as desired over a range of times. The same type of method also can be used to assess the efficacy of a therapeutic intervention in a subject where the therapy is being administered, or an ongoing therapy is changed.

In another embodiment, the invention provides a method for conducting a clinical trial to determine whether a candidate drug is effective in treating Parkinson's disease. The method is performed by analysing a biological sample from each subject in a population of subjects diagnosed with Parkinson's disease, and measuring the phosphorylation level of at least one of the biomarkers in the biological samples. Then, a dose of a candidate drug is administered to one portion or sub-population of the same subject population (“experimental group”) while a placebo is administered to the other members of the subject population (“control group”). After drug or placebo administration, a biological sample is acquired from the experimental and control groups and the same assays are performed on the biological samples as were previously performed to obtain phosphorylation measurement values. Depending upon the difference between the measured phosphorylation levels between the experimental and control groups, it can be seen whether the candidate drug is effective. The relative efficacy of two different drugs or other therapies for treating Parkinson's disease can be evaluated using this method by administering the drug or other therapy in place of the placebo. As will be apparent to one of skill in the art, the methods of the present invention may be used to evaluate an existing drug, being used to treat another indication, for its efficacy in treating Parkinson's disease (e.g., by comparing the efficacy of the drug relative to one currently used for treating Parkinson's disease in a clinical trial, as described above).

The present invention also provides molecules that specifically bind to the phosphorylated or unphosphorylated residue, or region comprising the residue, such as an antibody or antibody fragment. Such marker specific reagents have utility in isolating the markers and in detecting the presence of the markers, e.g., in immunoassays.

The present invention also provides kits for diagnosing Parkinson's disease, monitoring progression of the disease and assessing response to therapy, the kits comprising a container for a sample collected from a subject and at least one marker specific reagent.

In the present invention, the biomarkers may be used for diagnostic purposes. However, they may also be used for therapeutic, drug screening and patient stratification purposes (e.g., to group patients into a number of “subsets” for evaluation).

The present invention includes all methods relying on correlations between the biomarkers described herein and the presence of Parkinson's disease. In a preferred embodiment, the invention provides methods for determining whether a candidate drug is effective at treating Parkinson's disease by evaluating the effect it has on the biomarker values. In this context, the term “effective” is to be understood broadly to include reducing or alleviating the signs or symptoms of Parkinson's disease, improving the clinical course of the disease, decreasing the number or severity of exacerbations, or reducing any other objective or subjective indicia of the disease. Different drugs, doses and delivery routes can be evaluated by performing the method using different drug administration conditions. The method may also be used to compare the efficacy of two different drugs or other treatments or therapies for, Parkinson's disease

Phosphorylation levels (of the biomarkers) are to be understood as a measurement given from any stain or dye that recognises phosphor groups associated with proteins or peptides, for example Pro-Q Diamond or phosphor specific antibodies. The phosphorylation levels can also be measured after purification with different affinity columns such as IMAC or any other phosphor-binding surfaces.

It is expected that the levels of phosphorylation of the biomarkers described herein will be measured in combination with other signs, symptoms and clinical tests of Parkinson's disease, and/or other Parkinson's disease biomarkers reported in the literature. Likewise, more than one of the biomarkers of the present invention may be measured in combination. Measurement of the phosphorylation of the biomarkers of the invention along with any other markers known in the art, including those not specifically listed herein, falls within the scope of the present invention.

Because a diagnosis is rarely based exclusively on the results of a single test, the method may be used to determine whether a subject is more likely than not to have Parkinson's disease, or is more likely to have Parkinson's disease than to have another disease, based on the difference between the measured and standard level or reference range of the biomarker. Thus, for example, a patient with a putative diagnosis of Parkinson's disease may be diagnosed as being “more likely” or “less likely” to have Parkinson's disease in light of the information provided by a method of the present invention.

The biological sample may be of any tissue or fluid. Preferably, the sample is a CSF or serum sample, but other biological fluids or tissue may be used. Possible biological fluids include, but are not limited to, plasma, urine and neural tissue. A CSF biomarker in itself may be particularly useful for early diagnosis of disease. Furthermore, molecules initially identified in CSF may also be present, presumably at lower concentrations, in more easily obtainable fluids such as serum and urine. Such biomarkers may be valuable for monitoring all stages of the disease and response to therapy. Serum and urine also represent preferred biological samples as they are expected to be reflective of the systemic manifestations of the disease. In some embodiments, the level of a marker may be compared to the level of another marker or some other component in a different tissue, fluid or biological “compartment.” Thus, a differential comparison may be made of a marker in CSF and serum. It is also within the scope of the invention to compare the level of a marker with the level of another marker or some other component within the same compartment.

As will be apparent to those of ordinary skill in the art, the above description is not limited to making an initial diagnosis of Parkinson's disease, but also is applicable to confirming a provisional diagnosis of Parkinson's disease or “ruling out” such a diagnosis.

Phosphorylation measurements can be of (i) a biomarker of the present invention, (ii) a biomarker of the present invention and another factor known to be associated with Parkinson's disease (e.g., PET scan); (iii) a plurality of biomarkers comprising at least one biomarker of the present invention and at least one biomarker reported in the literature, or (iv) any combination of the foregoing. Furthermore, the amount of change in a biomarker level may be an indication of the relatively likelihood of the presence of the disease.

The present invention provides phosphorylated biomarkers that the present inventors have shown to be indicative of Parkinson's disease in a subject.

It is to be understood that any correlations between biological sample measurements of these biomarkers and Parkinson's disease, as used for diagnosis of the disease or evaluating drug effect, are within the scope of the present invention.

In the methods of the invention, phosphorylated biomarker levels are measured using conventional techniques. A wide variety of techniques are available, including mass spectrometry, chromatographic separations, 2-D gel separations, binding assays (e.g., immunoassays), competitive inhibition assays, and so on. Any effective method in the art for measuring the level of a protein or low molecular weight marker is included in the invention. It is within the ability of one of ordinary skill in the art to determine which method would be most appropriate for measuring a specific marker. Thus, for example, a robust ELISA assay may be best suited for use in a physician's office while a measurement requiring more sophisticated instrumentation may be best suited for use in a clinical laboratory. Regardless of the method selected, it is important that the measurements be reproducible.

the phosphorylated markers of the invention can be measured by mass spectrometry, which allows direct measurements of analytes with high sensitivity and reproducibility. A number of mass spectrometric methods are available and could be used to accomplish the measurement. Electrospray ionization (ESI), for example, allows quantification of differences in relative concentration of various species in one sample against another; absolute quantification is possible by normalization techniques (e.g., using an internal standard). Matrix-assisted laser desorption ionization (MALDI) or the related SELDI® technology (Ciphergen, Inc.) also could be used to make a determination of whether a marker was present, and the relative or absolute level of the marker. Moreover, mass spectrometers that allow time-of-flight (TOF) measurements have high accuracy and resolution and are able to measure low abundant species, even in complex matrices like serum or CSF.

For protein markers, quantification can be based on derivatization in combination with isotopic labelling, referred to as isotope coded affinity tags (“ICAT”)—In this and other related methods, a specific amino acid in two samples is differentially and isotopically labelled and subsequently separated from peptide background by solid phase capture, wash and release. The intensities of the molecules from the two sources with different isotopic labels can then be accurately quantified with respect to one another.

In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

In certain embodiments, the phosphorylated markers are measured using mass spectrometry in connection with a separation technology, such as liquid chromatography-mass spectrometry or gas chromatography-mass spectrometry. It is preferable to couple reverse-phase liquid chromatography to high resolution, high mass accuracy ESI time-of-flight (TOF) mass spectroscopy. This allows spectral intensity measurement of a large number of biomolecules from a relatively small amount of any complex biological material without sacrificing sensitivity or throughput. Analyzing a sample will allow the marker (specified by a specific retention time and m/z) to be determined and quantified.

As will be appreciated by one of skill in the art, many other separation technologies may be used in connection with mass spectrometry. For example, a vast array of separation columns is commercially available. In addition, separations may be performed using custom chromatographic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each component having a characteristic mass- to-charge ratio (m/z) and retention time (r.t). The presence of a peak with the m/z and retention time of a biomarker indicates that the marker is present. The peak representing a marker may be compared to a corresponding peak from another spectrum (e.g., from a control sample) to obtain a relative measurement. Any normalisation technique in the art (e.g., an internal standard) may be used when a quantitative measurement is desired. In addition, deconvoluting software is available to separate overlapping peaks. The retention time depends to some degree on the conditions employed in performing the liquid chromatography separation.

In other embodiments, the level of phosphorylation of the markers may be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the marker such as Affibody polypeptides). Standard protocols and data analysis are used to determine the marker concentrations from the assay data.

A number of the assays discussed above employ a reagent that specifically binds to the phosphorylated marker (“marker specific reagent”). Any molecule that is capable of specifically binding to a marker is included within the invention. In some embodiments, the marker specific reagents are antibodies or antibody fragments. In other embodiments, the marker specific reagents are non-antibody species. Thus, for example, a marker specific reagent may be an enzyme for which the marker is a substrate. The marker specific reagents may recognize any epitope of the targeted markers.

If phosphorylation of only one biomarker is measured, then that value must increase to indicate drug efficacy. If more than one biomarker is measured, then drug efficacy can be indicated by change in only one biomarker, all biomarkers, or any number in between. In some embodiments, multiple markers are measured, and drug efficacy is indicated by changes in multiple markers. Phosphorylation measurements can be of both biomarkers of the present invention and other measurements and factors associated with Parkinson's disease (e.g., measurement of biomarkers reported in the literature and/or other diagnostic techniques). Furthermore, the amount of change in a biomarker phosphorylation level may be an indication of the relatively efficacy of the drug.

In addition to determining whether a particular drug is effective in treating Parkinson's disease, biomarkers of the invention can also be used to examine dose effects of a candidate drug. There are a number of different ways that varying doses can be examined. For example, different doses of a drug can be administered to different subject populations, and phosphorylation measurements corresponding to each dose analyzed to determine if the differences in the inventive biomarkers before and after drug administration are significant. In this way, a minimal dose required to effect a change can be estimated. In addition, results from different doses can be compared with each other to determine how each biomarker behaves as a function of dose.

Analogously, administration routes of a particular drug can be examined. The drag can be administered differently to different subject populations, and phosphorylation measurements corresponding to each administration route analyzed to determined if the differences in the inventive biomarkers before and after drug administration are significant. Results from the different routes can also be compared with each other directly.

The present invention also provides kits for diagnosing Parkinson's disease, monitoring progression of the disease and assessing response to therapy. The kits comprise a container for sample collected from a patient and a marker specific reagent. In developing such kits, it is within the competence of one of ordinary skill in the art to perform validation studies that would use an optimal analytical platform for each marker. For a given marker, this may be an immunoassay or mass spectrometry assay. Kit development may require specific antibody development, evaluation of the influence (if any) of matrix constituent (“matrix effects”), and assay performance specifications. It may turn out that a combination of two or more markers provides the best specificity and sensitivity, and hence utility for monitoring the disease.

### DETAILED DESCRIPTION OF THE INVENTION

The present invention will now be further described with reference to the following figures which show:

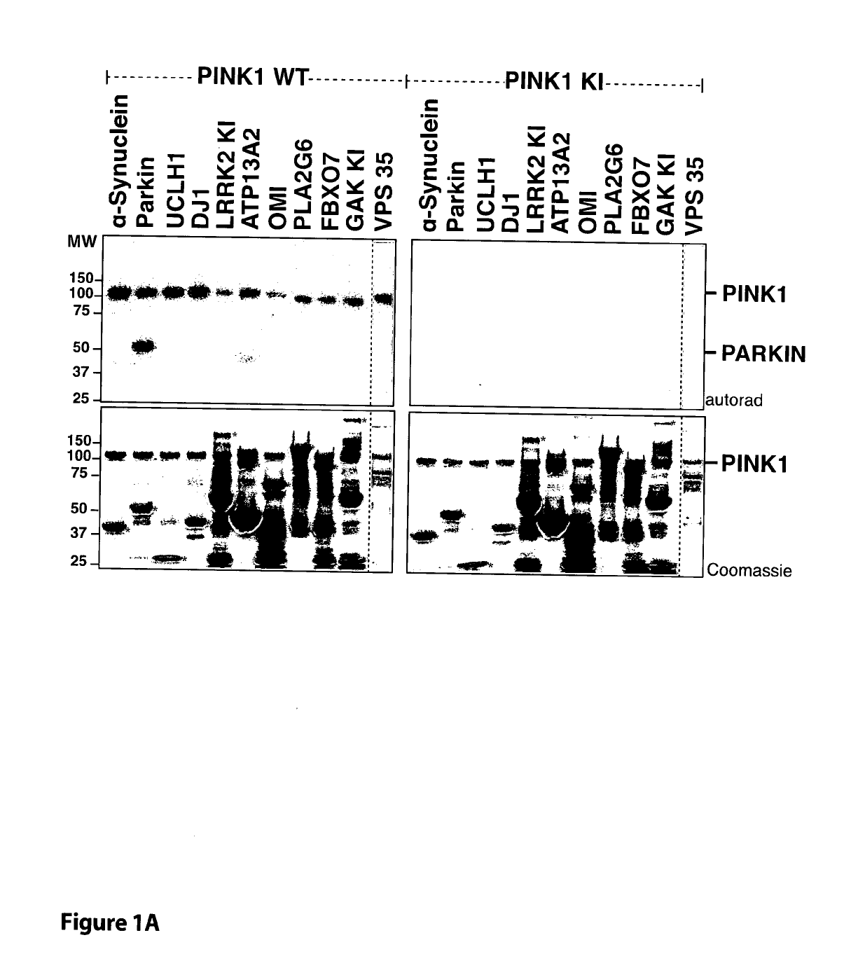

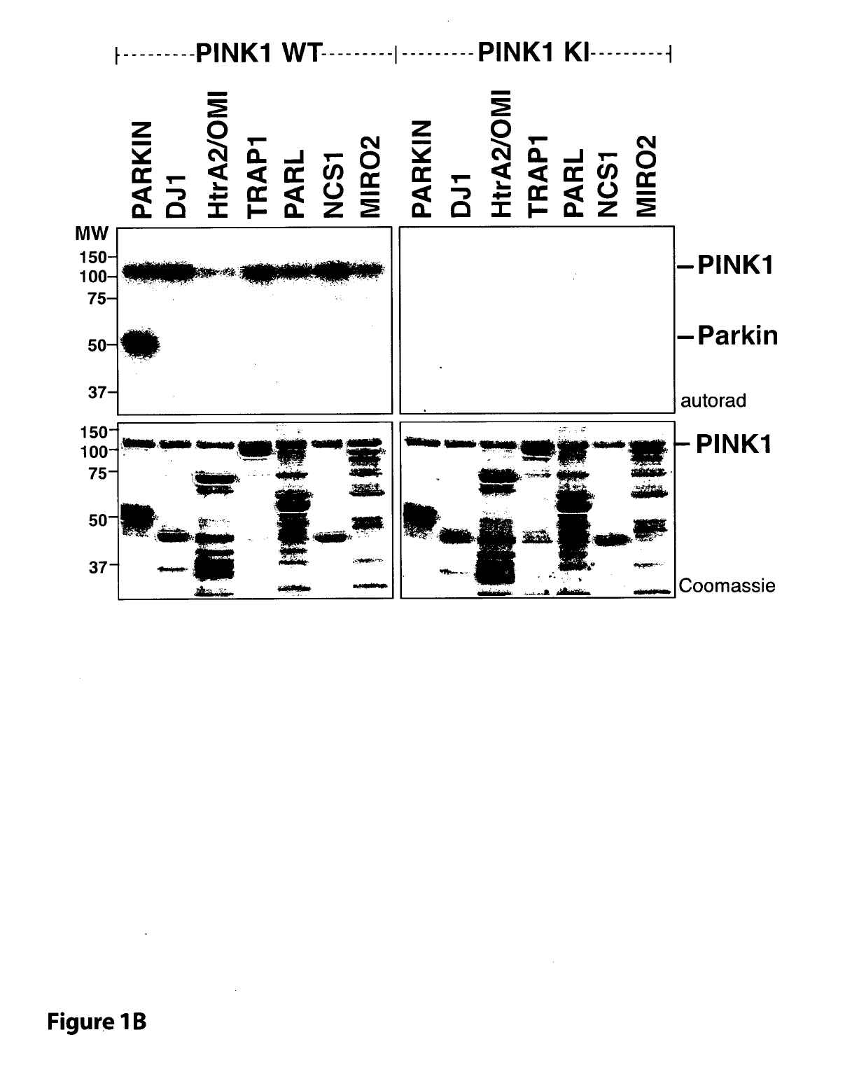

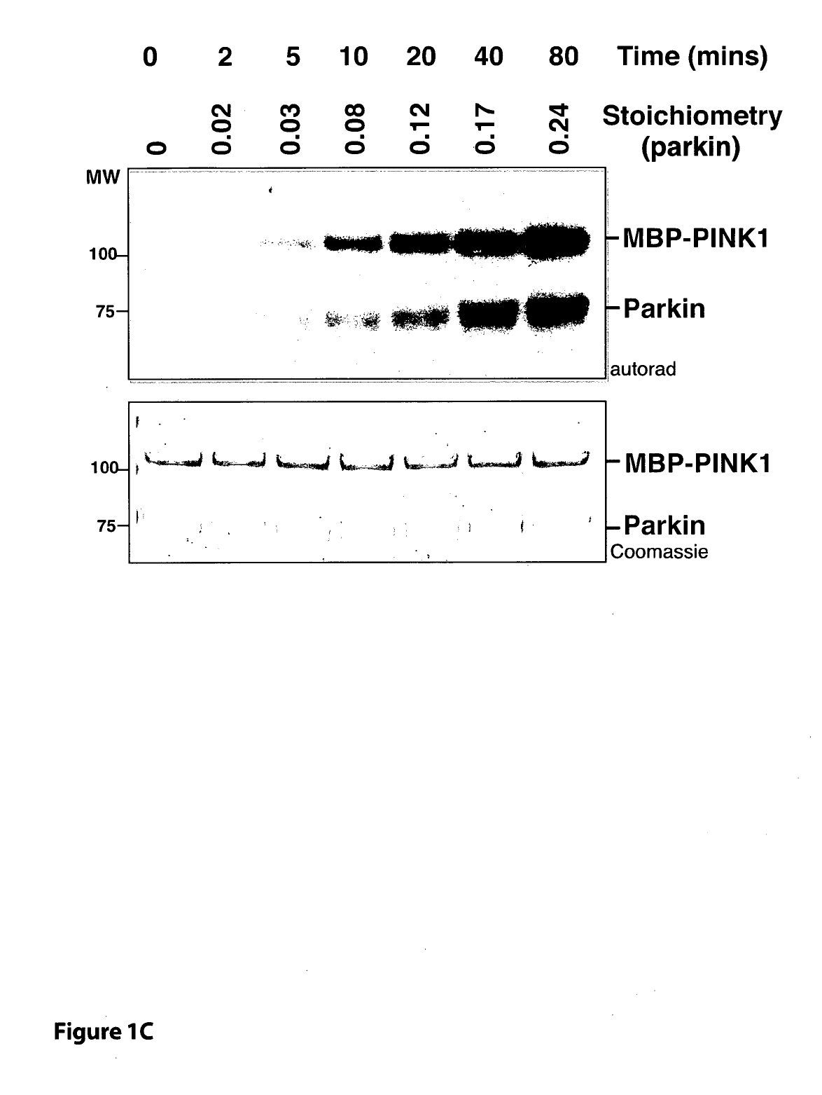

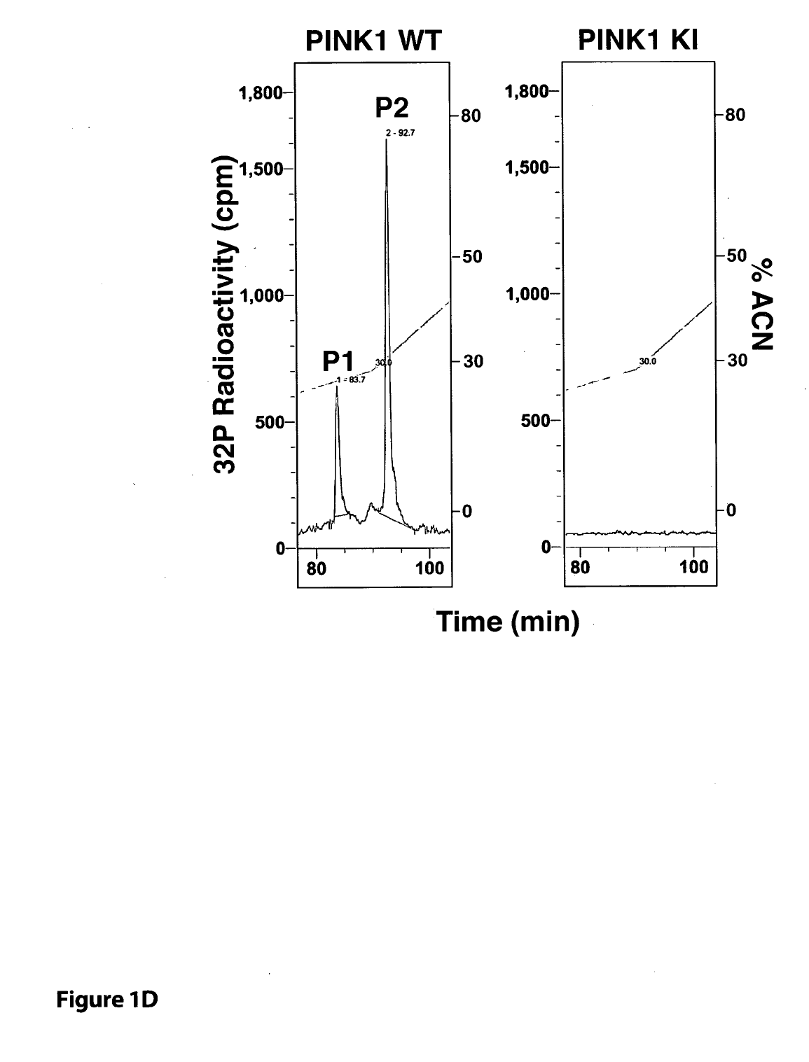

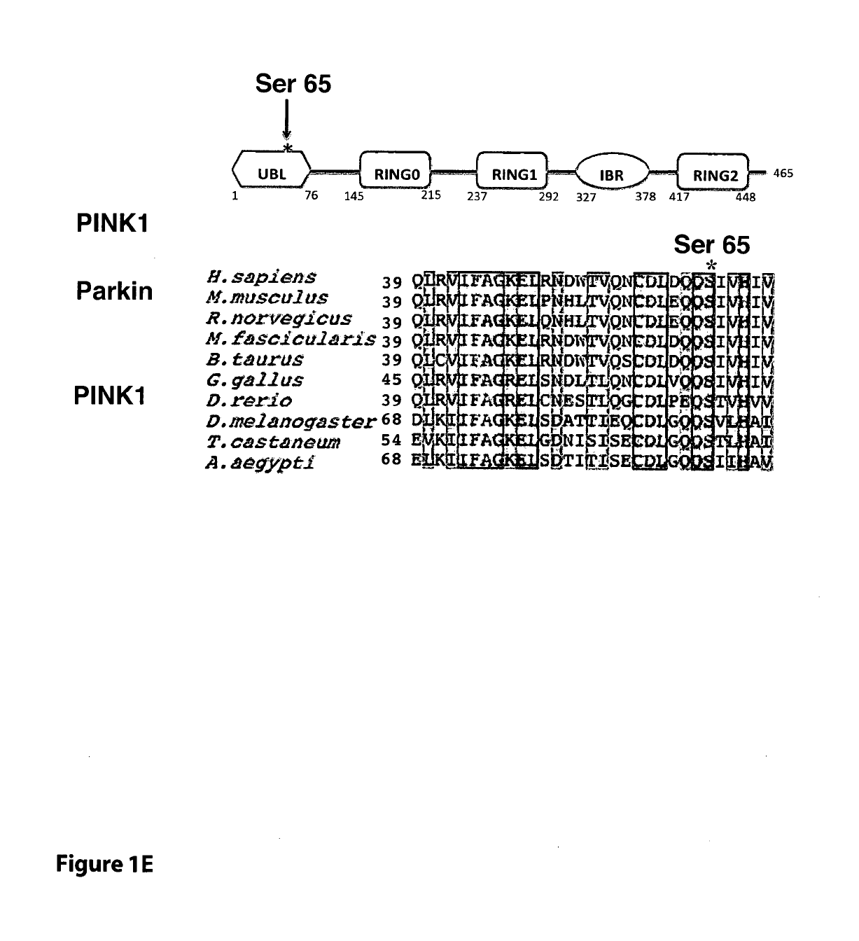

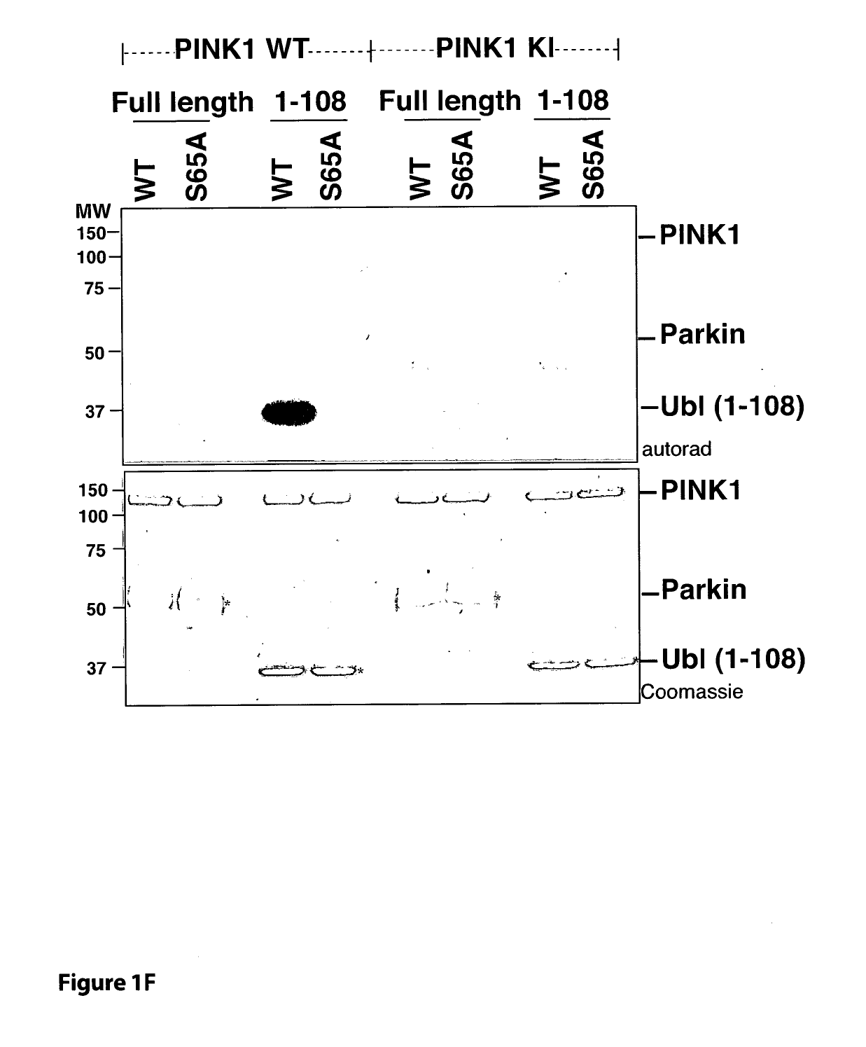

FIG. 1 TcPINK1 phosphorylates human parkin at Ser⁶⁵ in vitro. (A) The indicated Parkinson's disease-linked proteins (1 μM) were incubated with either full-length MBP-fusion of wild type TcPINK1 (1-570) or kinase inactive (KI) TcPINK1 (D359A) (0.5 μg) and [γ-³²P] ATP for 30 min. Assays were terminated by addition of SDS loading buffer and separated by SDS-PAGE. Proteins were detected by Colloidal Coomassie blue staining (upper panel) and incorporation of [γ-³²P] ATP was detected by autoradiography (lower panel). Similar results were obtained in three independent experiments. Fine dividing lines indicate that reactions were resolved on separate gels. The substrate bands on the Coomassie gel are denoted with a small red asterisk. All substrates were of human sequence and expressed in E. coli unless otherwise indicated. Tags on the substrates used for this experiment were GST-α-synuclein, parkin (no tag as His-SUMO tag cleaved off), His-UCHL1, GST-DJ1, GST-LRRK2 kinase inactive (1326-end D2017A), MBP-ATP13A2, GST-Omi, MBP-PLA2G6, GST-FBX07, GST-GAK-kinase-inactive (D191A), VPS35 (no tag as GST-tag cleaved off). (B) As in (A) except that proteins reported to interact with PINK1 were tested as PINK1 substrates. Human DJ1, Omi, TRAP1, PARL, NCS1 and Miro2 were expressed in E. coli with an N-terminal GST tag. Similar results were obtained in three independent experiments. (C) Time-course of phosphorylation of parkin by wild-type TcPINK1. MBP-TcPINK1 (0.5 μg) was incubated in the presence of GST-parkin (1 μg) and [γ-³²P] ATP for the times indicated and assays terminated by addition of SDS loading buffer. Samples were subjected to SDS-PAGE and proteins detected by Colloidal Coomassie blue staining (lower panel) and incorporation of [γ-³²P] ATP was detected by autoradiography (upper panel). Gel pieces were quantified by Cerenkov counting for calculation of the stoichiometry of parkin phosphorylation. Similar results were obtained in two independent experiments. (D) Mapping of phosphopeptides on parkin after phosphorylation by TcPINK1 in vitro. Full-length GST-parkin (1 μg) was incubated with 2 μg of either wild type TcPINK1 (1-570) or kinase inactive TcPINK1 (D359A) in the presence of Mg²⁺[γ-³²P] ATP for 60 min. Assays were terminated by addition of LDS loading buffer and separated by SDS-PAGE. Proteins were detected by Colloidal Coomassie blue staining and phosphorylated parkin was digested with trypsin. The resultant peptides were separated by reverse phase HPLC on a Vydac C₁₅ column (Vydac 218TP5215) equilibrated in 0.1% (v/v) trifluoroacetic acid and the column developed with an acetonitrile gradient (diagonal line). The flow rate was 0.2 ml/min and fractions (0.1 ml each) were collected and analysed for ³²P radioactivity by Cerenkov counting. Two major ³²P-labelled peaks (P1, P2) were identified following incubation with wild-type TcPINK1 (left). No peaks were identified following incubation with kinase-inactive TcPINK1 (right). (E) Schematic of domain organization of parkin illustrating that Ser⁶⁵ lies within the Ubl domain (upper panel) and sequence alignment of residues around Ser⁶⁵ in human parkin and a variety of lower organisms showing high degree of conservation. Abbreviations: Ubl, ubiquitin-like; IBR, in-between-RING; RING, really interesting new gene. (F) Mutation of Ser65Ala abolishes parkin phosphorylation by TcPINK1. Full-length wild type TcPINK1 (1-570) and kinase inactive TcPINK1 (D359A) against wild type or Ser65Ala mutants of full-length Parkin, or the isolated Ubl domain containing N-terminal fragment (aa 1-108). The indicated substrates (2 μM) were incubated in the presence of the indicated enzyme (1 μg) and [γ-³²P] ATP for 30 min. Assays were terminated by addition of SDS loading buffer and separated by SDS-PAGE. Proteins were detected by Colloidal Coomassie blue staining (lower panel) and incorporation of [γ-³²P] ATP was detected by autoradiography (upper panel).

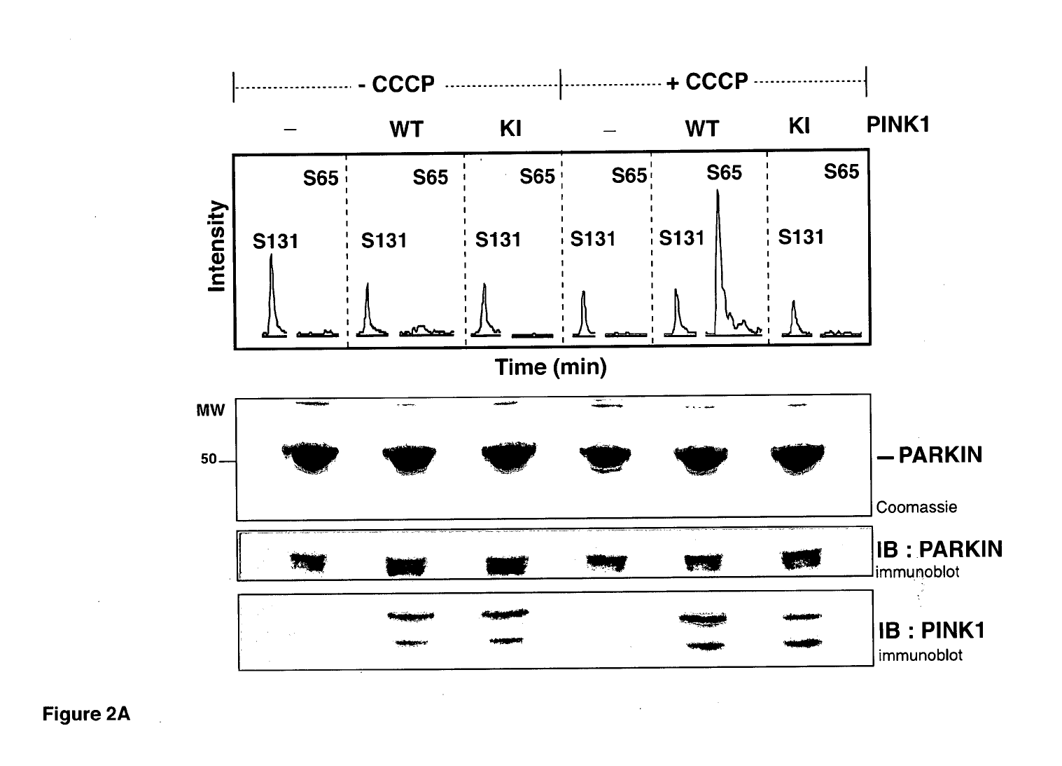

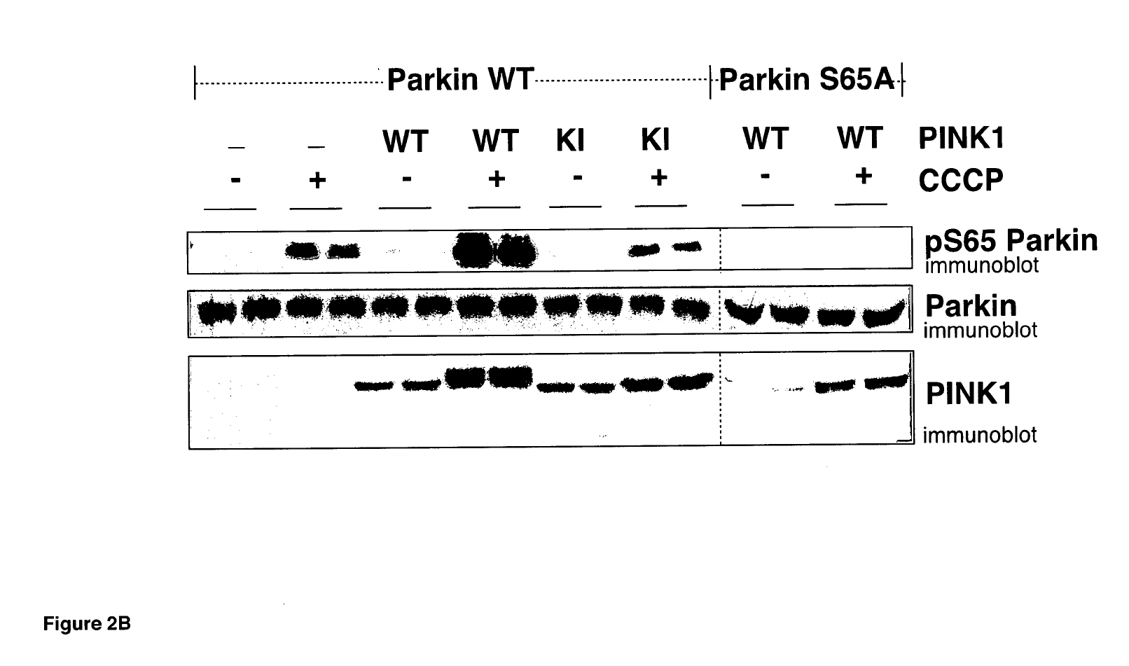

FIG. 2 Human parkin Ser⁶⁵ is a substrate of human PINK1 upon CCCP stimulation. (A) Confirmation by mass spectrometry that Ser⁶⁵ of human parkin is phosphorylated by CCCP-induced activation of human wild-type PINK1-FLAG.Flp-In T-Rex HEK293 cells expressing FLAG-empty, wild-type PINK1-FLAG, and kinase-inactive PINK1-FLAG (D384A) were co-transfected with HA-Parkin; induced with doxycycline and stimulated with 10 μM of CCCP for 3 hours. Whole cell extracts were obtained following lysis with 1% Triton and −30 mg of whole cell extract were subjected to immunoprecipitation with anti-HA-agarose and run on 10% SDS-PAGE and stained with colloidal Coomassie blue. Coomassie-stained bands migrating with the expected molecular mass of HA-parkin were excised from the gel, digested with trypsin, and subjected to LC-MS-MS on an LTQ-Orbitrap mass spectrometer. Extracted ion chromatogram analysis of Ser¹³¹ and Ser⁶⁵phosphopeptide (3⁺ R.NDWTVQNCDLDQQSIVHIVQRPWR.K+P). The total signal intensity of the phosphopeptide is plotted on the y-axis and retention time is plotted on the x-axis. The m/z value corresponding to the Ser¹³¹phosphopeptide was detected in all conditions whilst that of the Ser⁶⁵phosphopeptide was only detected in samples from wild-type PINK1-FLAG expressing cells following CCCP treatment. (B) Characterisation of parkin phospho-Ser⁶⁵ antibody. Flp-In T-Rex HEK293 cells expressing FLAG-empty, wild-type PINK1-FLAG, and kinase-inactive PINK1-FLAG were co-transfected with untagged wild-type (WT) or S65A mutant parkin; induced with doxycycline and stimulated with 10 μM of CCCP for 3 hours. 0.25 mg of 1% Triton whole cell lysate were subjected to immunoprecipitation with GST-Parkin antibody (S966C) covalently coupled to protein G Sepharose and then immunoblotted with anti-phospho-Ser⁶⁵ antibody in the presence of dephosphorylated peptide. 10% of the IP was immune-blotted with total anti-parkin antibody. 25 μg of whole cell lysate was immunoblotted with total PINK1 antibody. (C) In vitro phosphorylation of parkin by human PINK1 at Ser⁶⁵. Flp-In T-Rex HEK293 cells expressing wild-type PINK1-FLAG, and kinase-inactive PINK1-FLAG were induced to express protein by addition of 0.1 μg/ml of doxycycline in the culture medium for 24 hrs. Cells were then treated with 10 μg of CCCP for 3 hrs and lysates subjected to sub-cellular fractionation. 5 mg of mitochondrial lysate was subjected to immunoprecipitation with anti-FLAG agarose and utilized in an in vitro radioactive kinase assay with [γ-³²P]—Mg²⁺ ATP and E. coli expressed recombinant GST-parkin Ubl domain (aa1-108) [UBL] and mutant GST-parkin (aa 1-108) S65A [UBL S65A], purified from E. coli. One half of the assay reaction was run on a 10% SDS-PAGE and was subjected to autoradiography. Colloidal Coomassie stained gel shows equal loading of recombinant substrate. The other half of the reaction was immunoblotted with anti-phospho-Thr²⁵⁷ PINK1 and total PINK1 antibodies following 8% SDS-PAGE.

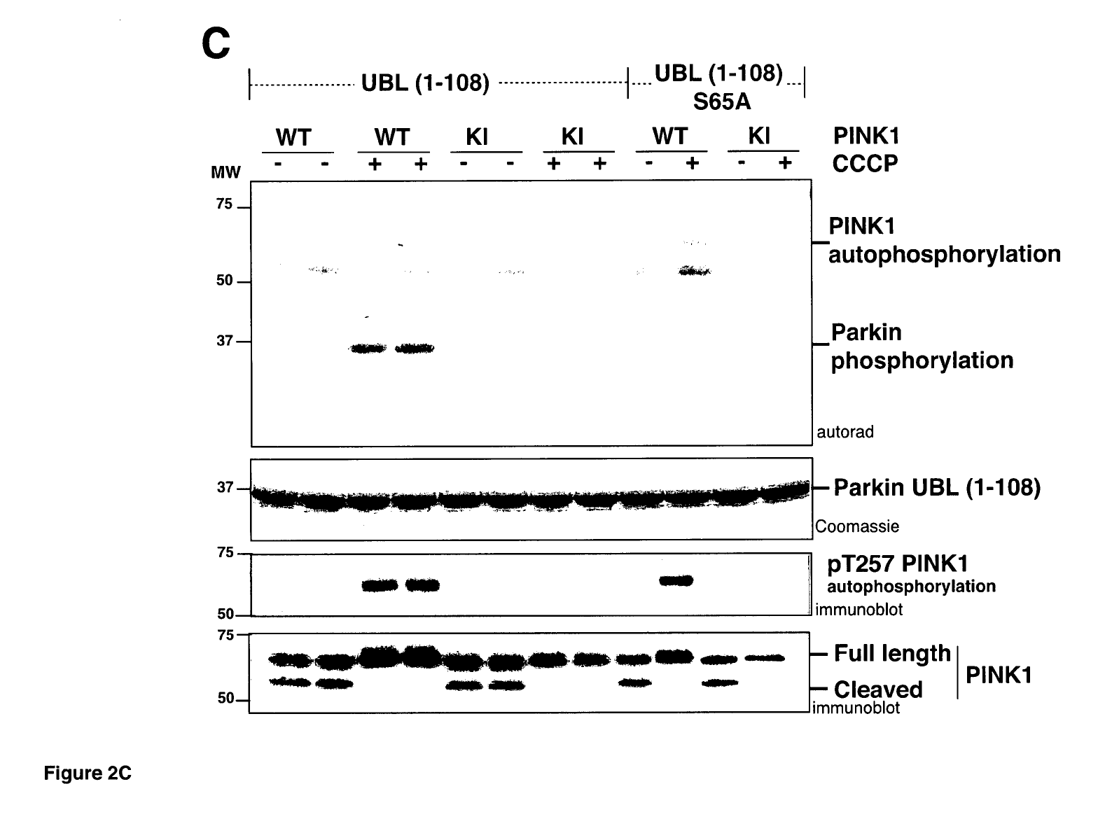

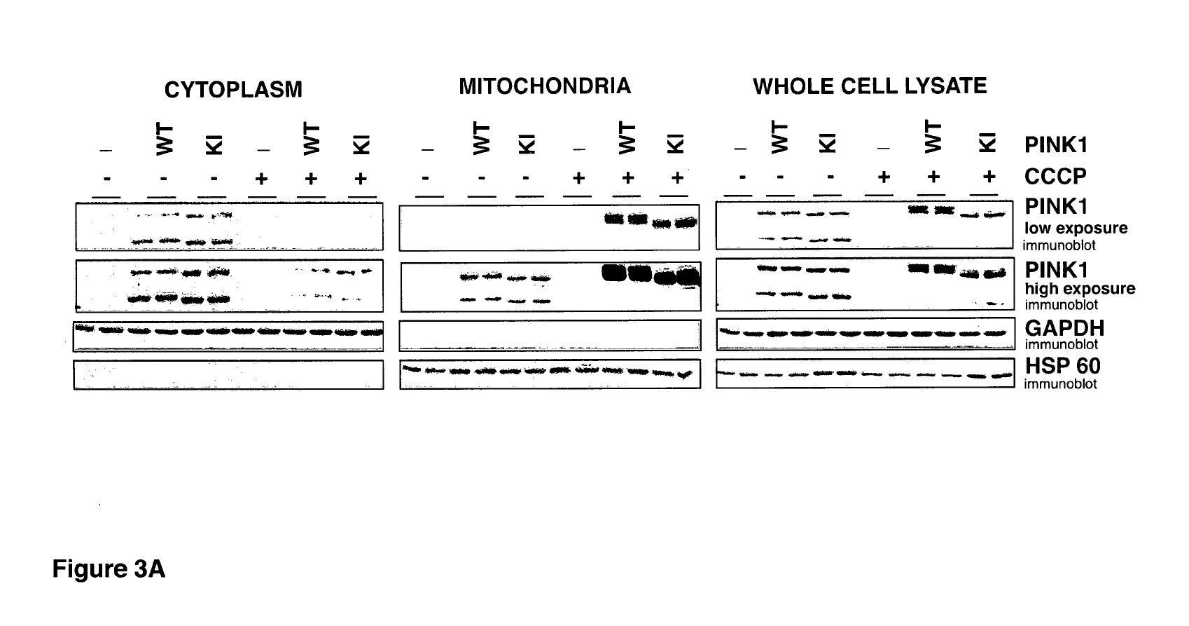

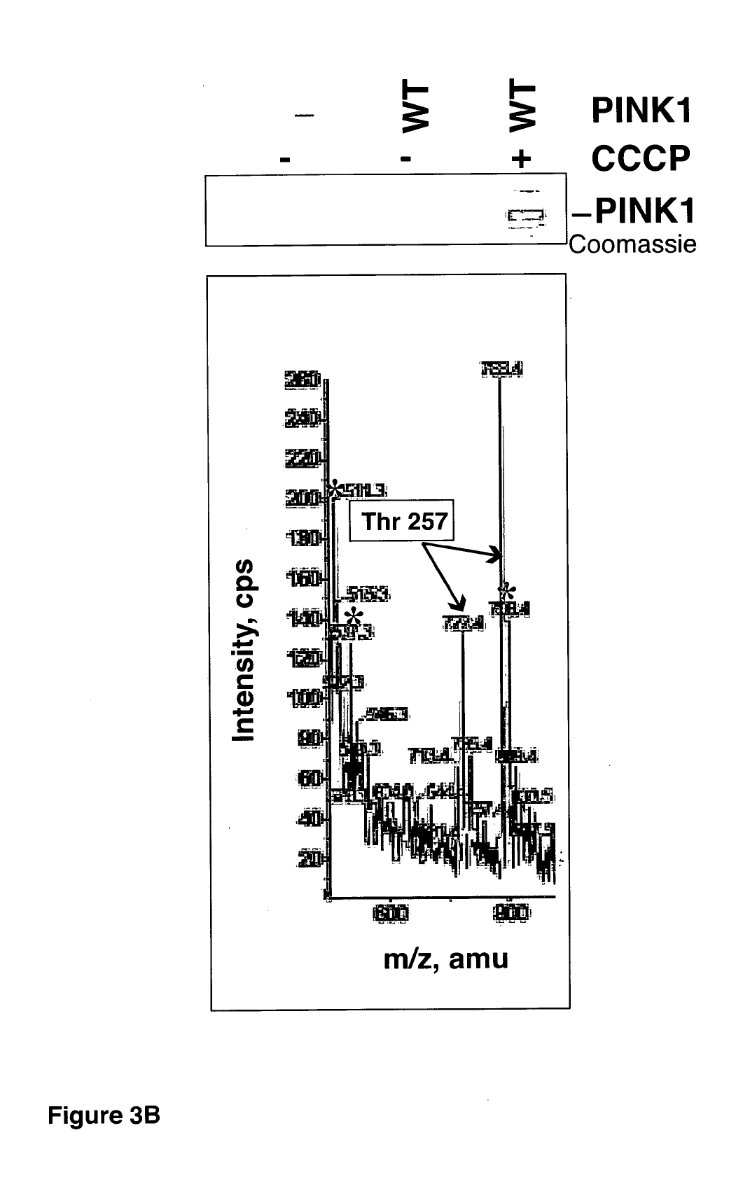

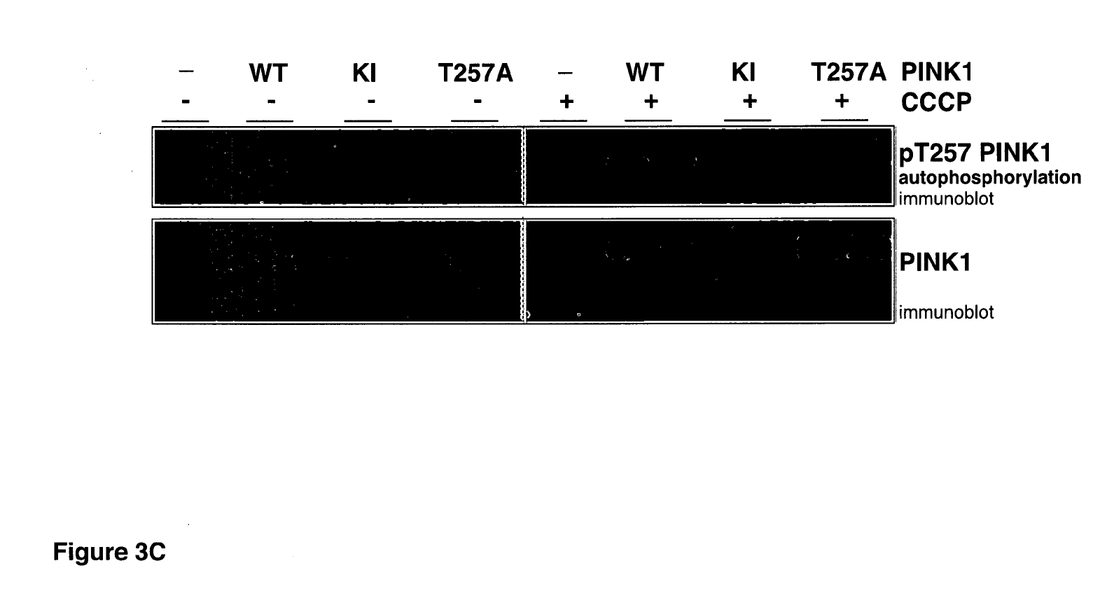

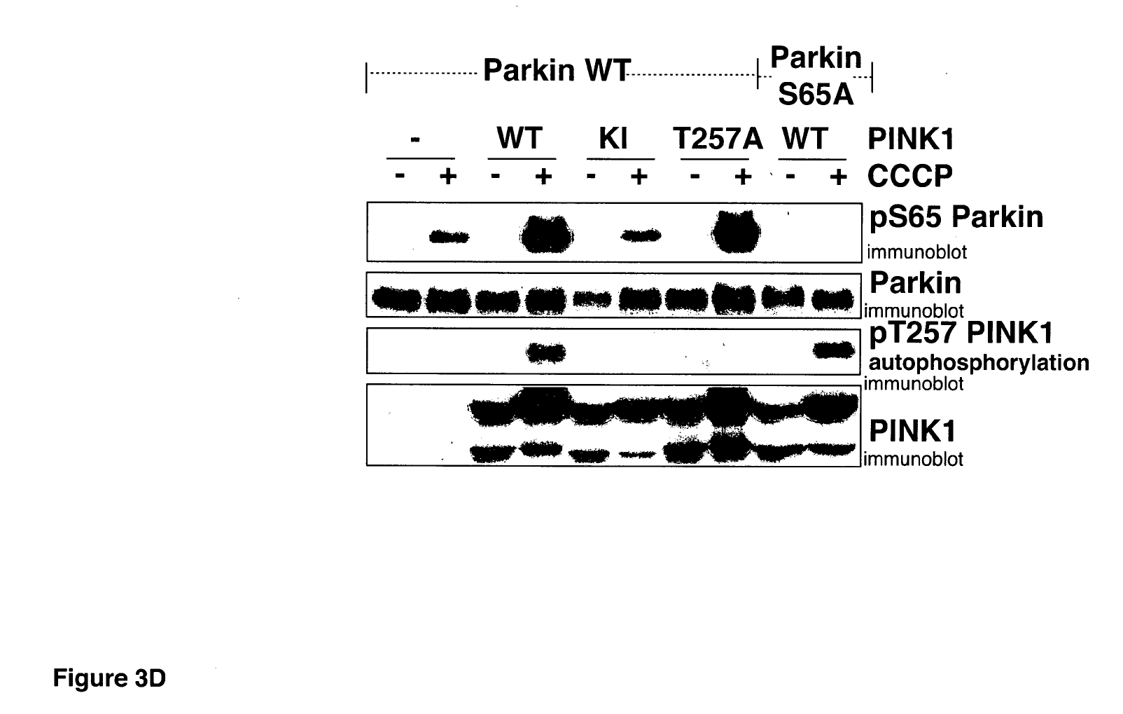

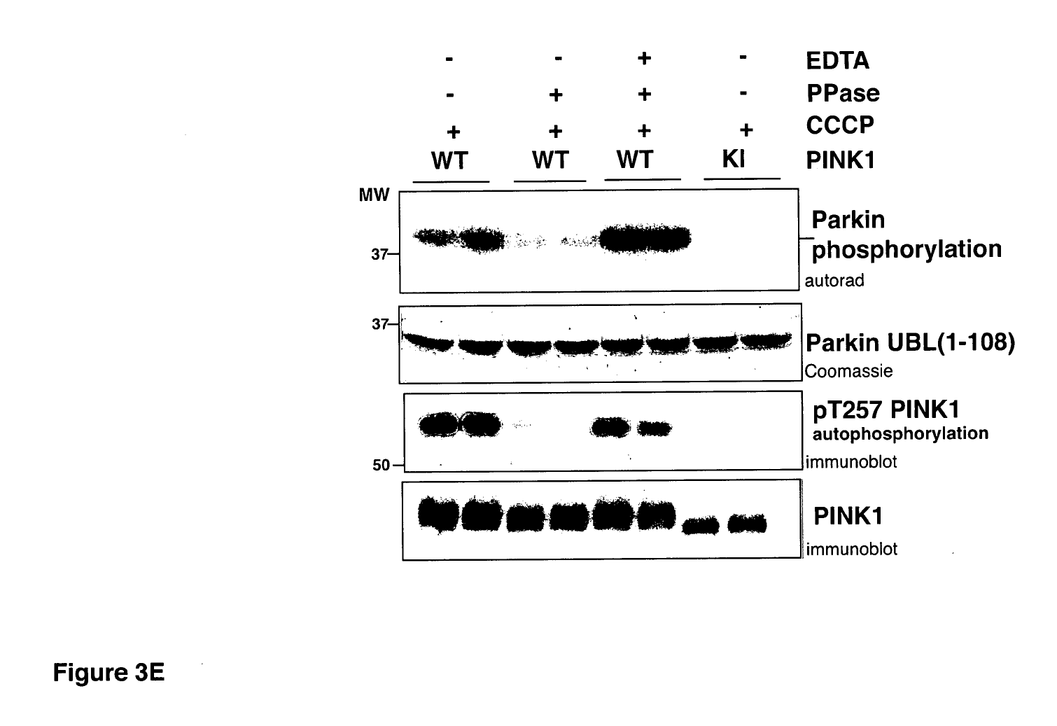

FIG. 3 Identification and characterization of a novel autophosphorylation site of PINK1 induced by the mitochondrial uncoupling agent CCCP. (A) CCCP induces a bandshift in wild-type but not kinase-inactive PINK1. Flp-In T-Rex HEK 293 cell line stably expressing FLAG alone, wild-type or kinase-inactive PINK1-FLAG were induced to express protein by addition of 0.1 μg/ml of doxycycline in the culture medium for 24 hrs. Cells were then treated with 10 μg of CCCP for 3 hrs and lysates subjected to sub-cellular fractionation. 25 μg of cytoplasmic or mitochondrial lysate were resolved by 8% SDS-PAGE. Relative purity of the fractions was confirmed using cytoplasmic and mitochondrial markers namely GAPDH and HSP60 respectively. Whole cell extracts from the same cells were also made in parallel using 1% Triton lysis as described in the methods. In mitochondrial and whole cell extracts, both wild-type and kinase-inactive PINK1 became stabilized by CCCP but a bandshift was noted for wild-type PINK1 which was revealed to be a doublet on lower exposure. The upper band was absent from kinase-inactive PINK1 treated with CCCP. (B) Identification of Thr²⁵⁷ phosphorylation site on PINK1. Flp-In T-Rex HEK 293 cell line stably expressing FLAG alone, or wild-type PINK1-FLAG were treated with DMSO or 10 μg of CCCP for 3 hours. Recombinant PINK1 was immunoprecipitated from 10 mg of mitochondrial extract for each condition using anti-FLAG-agarose and subjected to 4-12% gradient SDS-PAGE and stained with colloidal Coomassie blue. The Coomassie-stained bands migrating with the expected molecular mass of PINK1-FLAG were excised from the gel, digested with trypsin, and subjected to precursor-ion scanning mass spectroscopy. The major phosphopeptide that is indicated “Thr257” was seen from cells expressing wild-type PINK1-FLAG treated with CCCP and this was not seen in bands from the other 2 conditions. The figure shows the signal intensity (cps, counts of ions per second) of the HPO₃⁻ ion (−79 Da) seen in negative precursor ion scanning mode versus the ion distribution (m/z) for the Thr²⁵⁷ phosphopeptide. The observed values of 722.4 and 788.4 are for the VALAGEYGAVTYR and VALAGEYGAVTYRK variants respectively of the Thr²⁵⁷ peptide as [M-2H]²⁻ ions. Other phosphopeptides marked with an asterisk were observed but we were unable to assign phosphorylation site(s). (C) Evidence that CCCP induces PINK1 auto-phosphorylation using a phospho-specific Thr²⁵⁷ antibody. 0.5 mg of mitochondrial extracts (treated with DMSO or 10 μg of CCCP for 3 hours) of Flp-In T-Rex stable cell lines expressing FLAG empty, wild-type PINK1-FLAG, kinase-inactive PINK1-FLAG (D384A) and phospho-mutant T257A were immunoprecipitated with anti-FLAG agarose and resolved by 8% SDS-PAGE. Blots were probed with pT257 PINK1 phospho antibody and anti-PINK1 antibody. (D) Mutation of Thr257Ala PINK1 does not affect parkin Ser⁶⁵ phosphorylation. Flp-In T-Rex HEK293 cells expressing FLAG-empty, wild-type PINK1-FLAG, kinase-inactive PINK1-FLAG and T257A PINK1-FLAG were co-transfected with untagged wild-type (WT) or S65A mutant parkin; induced with doxycycline and stimulated with 10 μg of CCCP for 3 hours. 0.25 mg of 1% Triton whole cell lysate were subjected to immunoprecipitation with GST-Parkin antibody (S966C) covalently coupled to protein G Sepharose and then immunoblotted with anti-phospho-Ser⁶⁵ antibody in the presence of dephosphorylated peptide. 10% of the IP was immune-blotted with total anti-parkin antibody. 1 mg of 1% Triton whole cell lysate were immunoprecipitated with anti-FLAG agarose and resolved by 8% SDS-PAGE. Blots were probed with pT257 PINK1 phospho antibody and anti-PINK1 antibody. (E) PINK1 dephosphorylation by lambda phosphatase inhibits PINK1 activity. C-terminal-FLAG tagged wild-type or kinase-inactive (D384A) PINK1 were immunoprecipitated from 5 mg of mitochondrial enriched extracts using anti-FLAG agarose beads. Wild-type PINK1 was incubated with or without 1000U of Lambda phosphatase or treated with lambda phosphatase along with 50 mM EDTA. Kinase-inactive PINK1 was incubated in buffer alone without lambda phosphatase. The beads were washed three times in 50 mM Tris pH 7.5, 0.1 mM EGTA and then utilized in an in vitro kinase assay with GST-parkin UBL (1-108) as the substrate. Samples were analyzed as described in Legend to FIG. 1.

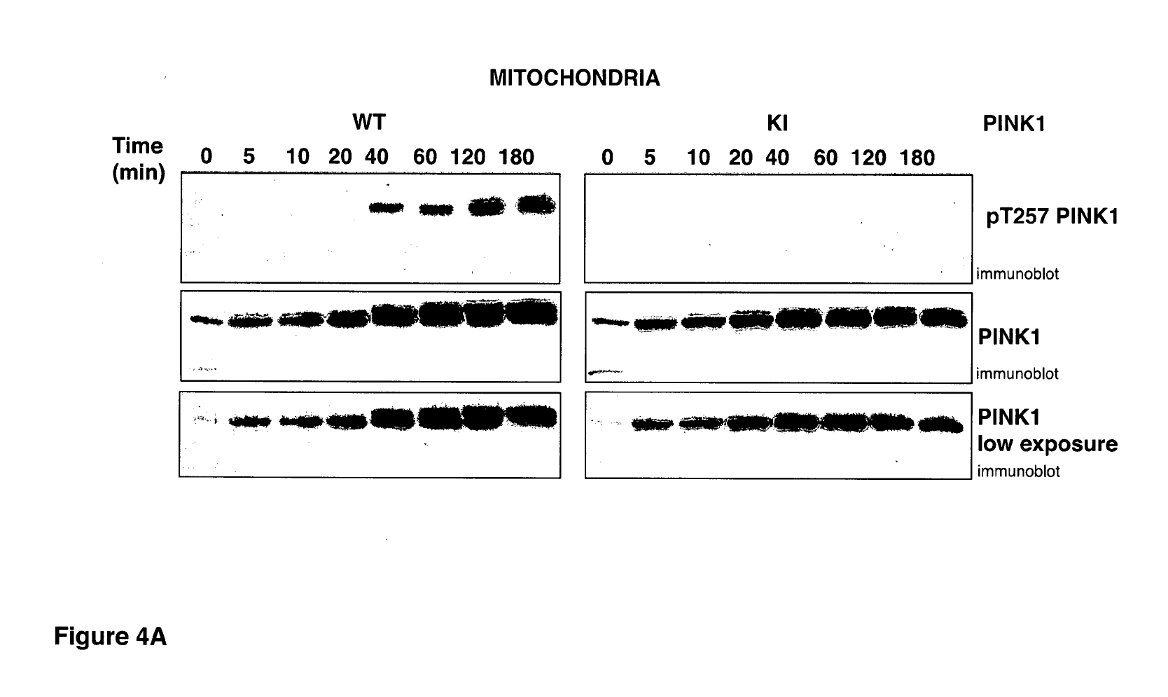

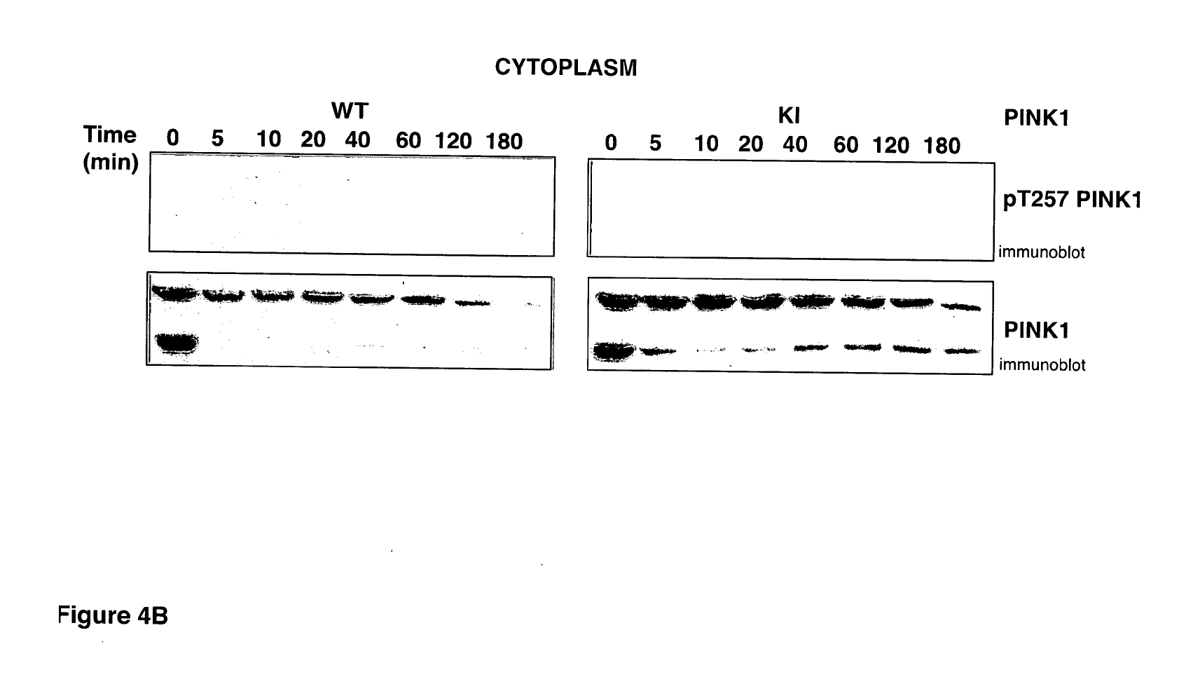

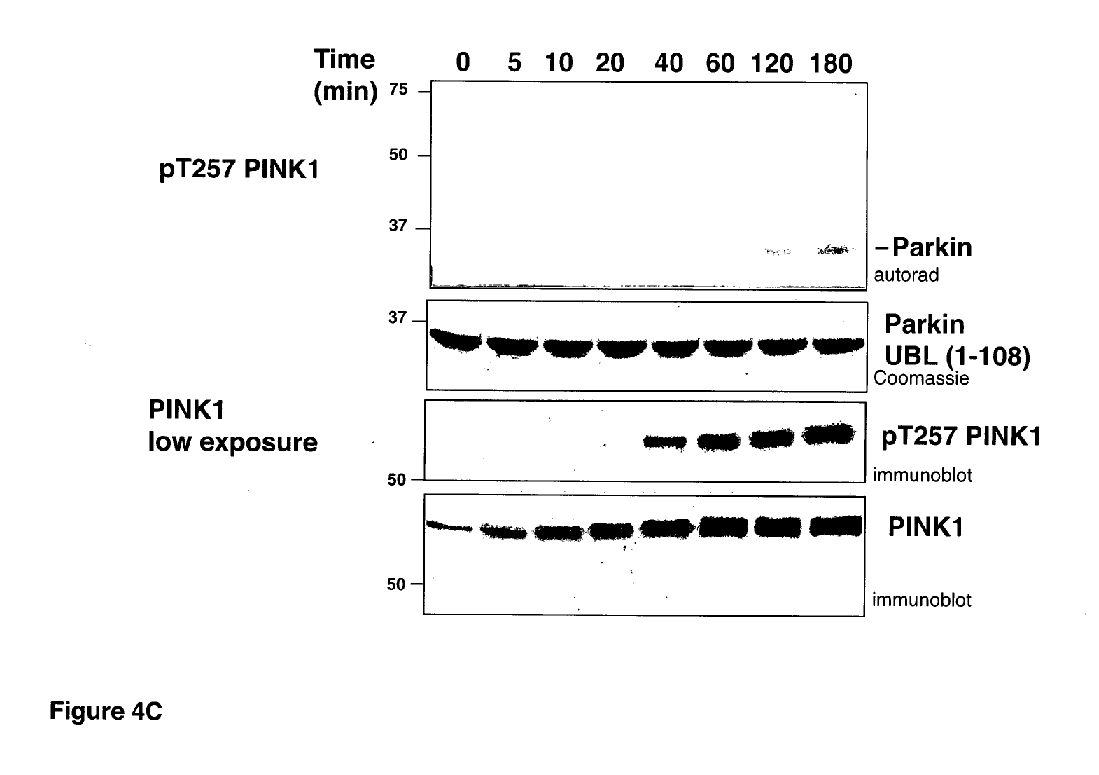

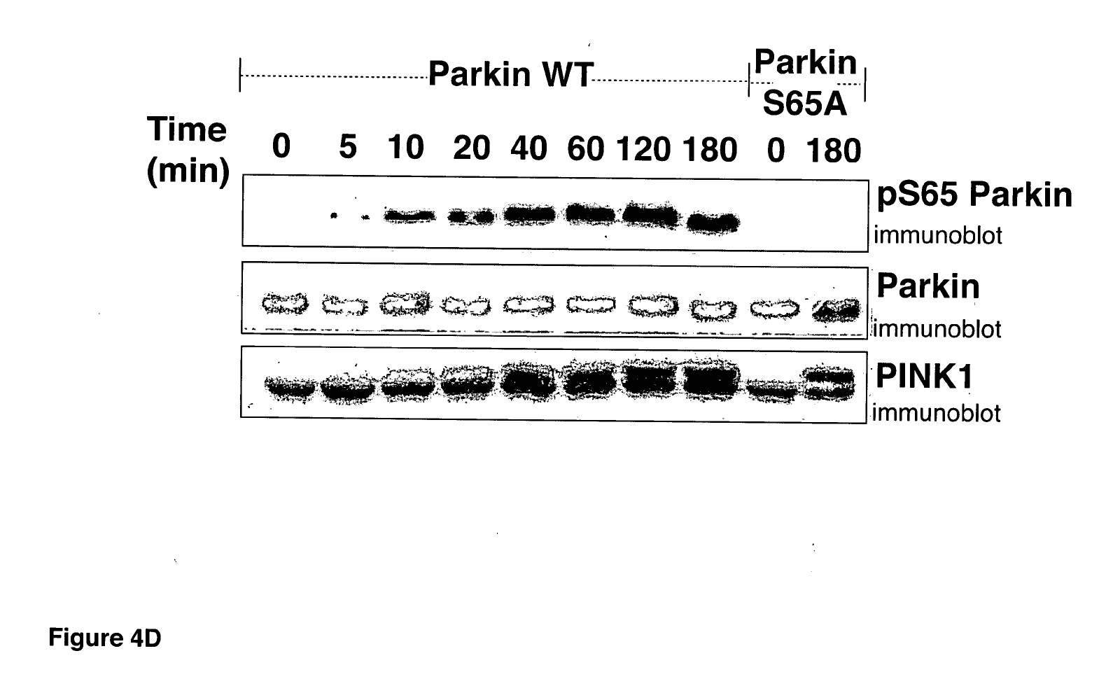

FIG. 4 Time course of CCCP-induced activation of PINK1. (A) Timecourse of PINK1 autophosphorylation in vivo. Flp-In TRex HEK 293 cells stably expressing PINK1-FLAG wild-type and kinase-inactive (D384A) were stimulated at the indicated timepoints with 10 μg of CCCP. 0.5 mg of mitochondrial extracts were immunoprecipitated with anti-FLAG agarose and resolved by 8% SDS-PAGE. Immunoblotting was performed with anti-phospho-Thr²⁷⁵ antibody or total PINK1. (B) No time-dependent activation of cytoplasmic PINK1 in vivo. As in (A) cytoplasmic extracts were obtained at the indicated time-points and immunoprecipitated with anti-FLAG agarose and resolved by 8% SDS-PAGE. Immunoblotting was performed with PINK1 anti-phospho-Thr²⁷⁵ antibody or total PINK1 antibody. (C) Timecourse of PINK1 activation in vitro. Flp-In T-Rex HEK293 cells expressing wild-type PINK1-FLAG were stimulated for indicated time-points. 5 mg of mitochondrial lysate were subjected to immunoprecipitation with anti-FLAG agarose and utilized in an in vitro radioactive kinase assay with [γ-³²P]—Mg²⁺ ATP and E. coli expressed recombinant GST-parkin fragment (aa1-108) [UBL] purified from E. coli. One half of the assay reaction was run on a 10% SDS-PAGE and was subjected to autoradiography. Colloidal Coomassie stained gel shows equal loading of recombinant substrate. The other half of the reaction was immunoblotted with anti-phospho-Thr²⁵⁷ PINK1 and total PINK1 antibodies following 8% SDS-PAGE. (D) Timecourse of parkinSer⁶⁵ phosphorylationin vivo. Flp-In TRex HEK 293 cells stably expressing wild-type PINK1-FLAG were co-transfected with untagged wild-type (WT) or S65A mutant parkin; induced with doxycycline and stimulated with 10 μg of CCCP at the indicated timepoints. 0.25 mg of 1% Triton whole cell lysate were subjected to immunoprecipitation with GST-Parkin antibody (S966C) covalently coupled to protein G Sepharose and then immunoblotted with anti-phospho-Ser⁶⁵ antibody in the presence of dephosphorylated peptide. 10% of the IP was immune-blotted with total anti-parkin antibody. 25 μg of whole cell lysate was immunoblotted with total PINK1 antibody.

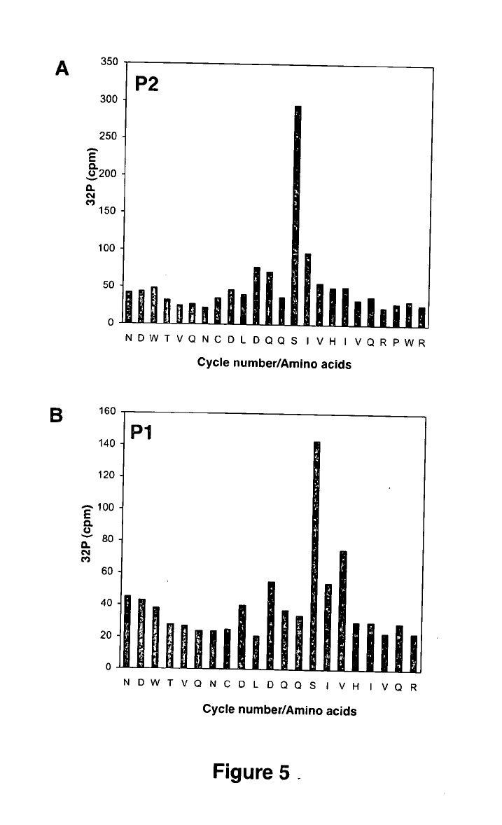

FIG. 5 Identification of the Ser⁶⁵ phosphorylation site by Edman sequencing and mass spectrometry. Phosphopeptides P2 (A) and P1 (B) from FIG. 1C were sequenced by solid-phase Edman degradation using an Applied Biosystems 494C sequencer after the peptides were coupled to Sequelon-arylamine membrane (Applied Biosystems) as described previously (Campbell and Morrice 2002). The amino acid sequence deduced from the LC-MS-MS analysis is shown using the single-letter code for amino acids.

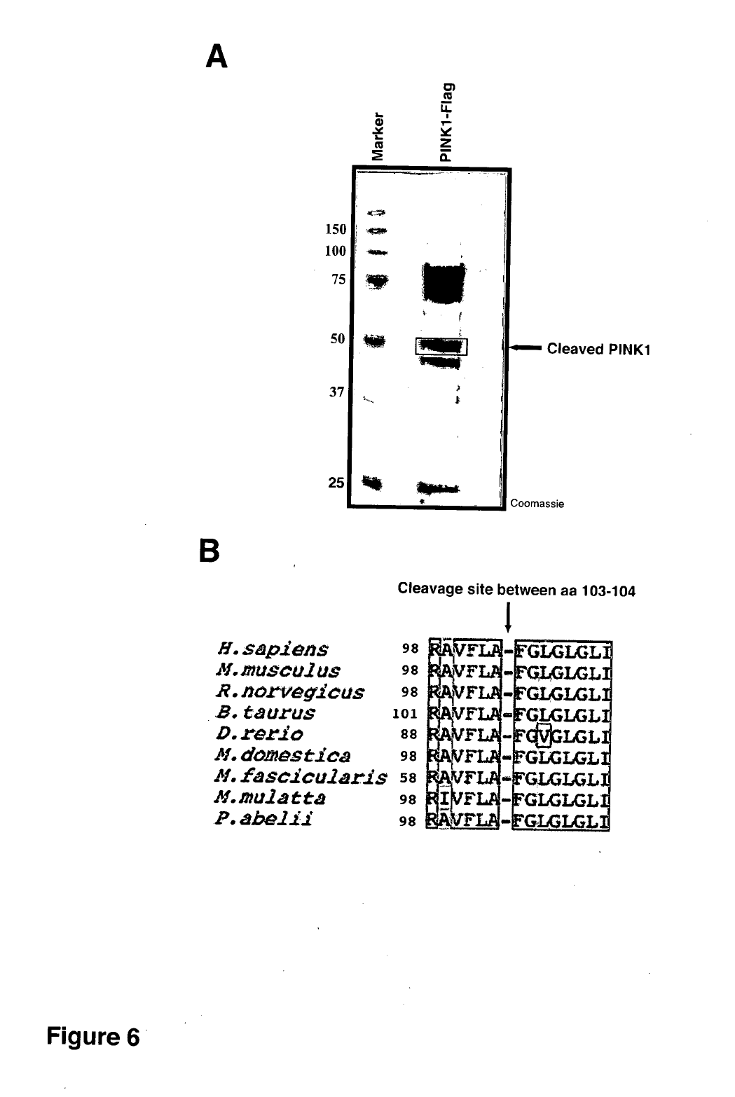

FIG. 6 Mapping of PINK1 cleavage site by N-terminal Edman sequencing. HEK293 cells were transiently transfected with wild-type PINK1-FLAG and 100 mg of whole cell lysate immunoprecipitated with anti-FLAG agarose. After electrophoresis, samples were transferred to Immobilon PVDF membrane and stained with Coomassie Blue. (A) Coomassie stained PVDF membrane showing band corresponding to the cleaved form of PINK1 that was excised and subjected to Edman degradation and analysis. The amino acid sequence obtained in the gel band started with FGLGLG (residues 104-109). Representative of 3 independent experiments. (B) Sequence alignment of residues around Phe¹⁰⁴ in human PINK1 showing high degree of conservation amongst higher organisms. Cleavage site indicated by an arrow.

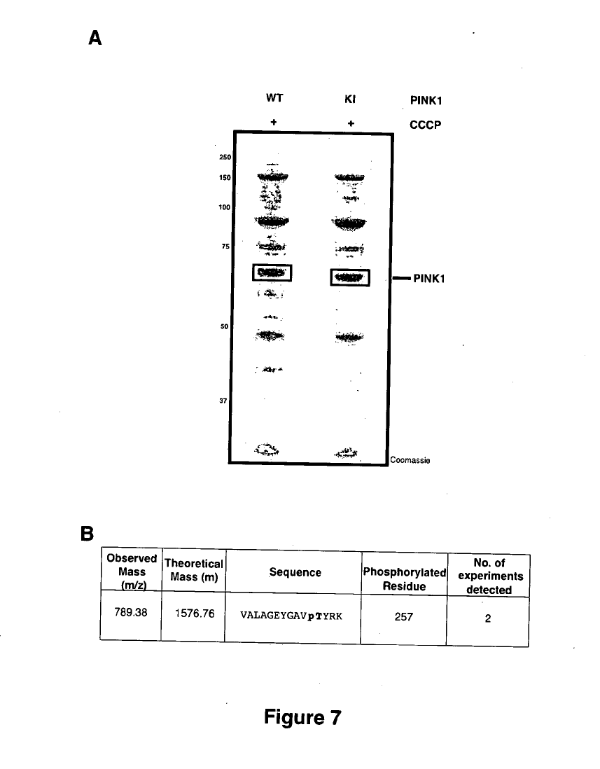

FIG. 7 Mass spectrometry confirmation that phosphorylation of PINK1 Thr²⁵⁷ is an autophosphorylation site.

Flp-In T-Rex HEK 293 cell line stably expressing wild-type or kinase-inactive PINK1-FLAG (D384A) were treated 10 μg of CCCP for 3 hrs. (A) Recombinant PINK1 was immunoprecipitated from 10 mg of mitochondrial extract for each condition using anti-FLAG-agarose, subjected to 4-12% gradient SDS-PAGE, and stained with colloidal Coomassie blue. (B) The Coomassie-stained bands migrating with the expected molecular mass of PINK1-FLAG were excised from the gel, digested with trypsin, and subjected to LC-MS-MS on an LTQ-Orbitrap mass spectrometer. The Thr²⁵⁷phosphopeptide was only detected in the wild-type PINK1-FLAG band.

### MATERIALS AND METHODS

### Reagents and General Methods

Tissue culture reagents were from Life Technologies. [γ-³²P] ATP was from Perkin Elmer. The Flp-In T-Rex HEK 293 cell line was from Invitrogen and stable cell lines were generated according to the manufacturer's instructions by selection with hygromycin. Restriction enzyme digests, DNA ligations and other recombinant DNA procedures were performed using standard protocols. All mutagenesis was carried out using the QuikChange® site-directed-mutagenesis method (Stratagene) with KOD polymerase (Novagen). All DNA constructs were verified by DNA sequencing, which was performed by The Sequencing Service, School of Life Sciences, University of Dundee, using DYEnamic ET terminator chemistry (Amersham Biosciences) on Applied Biosystems automated DNA sequencers. DNA for mammalian cell transfection were amplified in E. coli DH5α strain and plasmid preparation was done using Qiagen Maxi prep Kit according to manufacturers protocol. DNA for bacterial protein expression were transformed in E. coli BL21 DE3 RIL (codon plus) cells (Stratagene).

### Cell Culture and Stimulation

Flp-In T-Rex stable cell lines were cultured using DMEM (Dulbecco's modified Eagle's medium) supplemented with 10% FBS (Fetal Bovine Serum), 2 mM L-Glutamine, 1× Pen/Strep, 15 μg/ml of Blasticidin and 100 μg/ml of Hygromycin. Cell transfections of HA-parkin or untagged parkin were performed using the polyethyleneimine (PEI) method (Reed et al, 2006). Cultures were induced to express protein by addition of 0.1 μg/ml of Doxycycline in the medium for 24 hours. To uncouple mitochondria, cells were treated with 10 μM CCCP (Sigma) dissolved in DMSO for the indicated times. An equivalent volume of DMSO was used as a control.

### Buffers and Methods for Mammalian Cell Lysis

Cells were lysed and fractionated by the indicated buffer and methods: Whole cell lysis using buffer: 50 mM Tris/HCl (pH 7.4), 1 mM EGTA, 1 mM EDTA, 1%(w/v) 1 mM sodium orthovanadate, 10 mM sodium β-glycerophosphate, 50 mM NaF, 5 mM sodium pyrophosphate, 0.27M sucrose, 1 mM benzamidine and 2mMPMSF and 1%(v/v) Triton X-100. Lysates were clarified by centrifugation at 13,000 rpm for 10 min at 4° C. and the supernatant was collected. Mitochondrial fractionation: cells were lysed in buffer containing 250 mM sucrose, 20 mM HEPES, 3 mM EDTA, 1%(w/v) 1 mM sodium orthovanadate, 10 mM sodium β-glycerophosphate, 50 mM NaF, 5 mM sodium pyrophosphate, pH 7.5 and protease inhibitor cocktail (Roche) at 4° C. Cells were disrupted using a glass hand held homogeniser (20 passes) and the lysate was clarified by centrifuging for 10 min at 800 g at 4° C. The supernatant was further centrifuged at 16,600 g for 10 min. The resultant supernatant served as the cytosolic fraction. The pellet containing the mitochondrial fraction was resuspended in buffer containing 1% Triton X-100 and centrifuged at 13,000 rpm for 10 min. This supernatant contained solubilized mitochondrial proteins. All lysates were snap-frozen at −80° C. until use. Protein concentration was determined using the Bradford method (Thermo Scientific) with BSA as the standard.

Buffers for E. coli Protein Purification

Lysis buffer contained 50 mM Tris-HCl (pH 7.5), 150 mM NaCl, 1 mM EDTA, 1 mM EGTA, 5% (v/v) glycerol, 1% (v/v) Triton X-100, 0.1% (v/v) 2-mercaptoethanol, 1 mM benzamidine and 0.1 mM phenylmethylsulfonyl fluoride (PMSF). Wash buffer contained 50 mM Tris-HCl (pH 7.5), 500 mM NaCl, 0.1 mM EGTA, 5% (v/v) glycerol, 0.03% (v/v) Brij-35, 0.1% (v/v) 2-mercaptoethanol, 1 mM benzamidine and 0.1 mM PMSF. Equilibration buffer contained 50 mM Tris-HCl (pH 7.5), 150 mM NaCl, 0.1 mM EGTA, 5% (v/v) glycerol, 0.03% (v/v) Brij-35, 0.1% (v/v) 2-mercaptoethanol, 1 mM benzamidine and 0.1 mM PMSF. Elution buffer was equilibration buffer with the addition of 12 mM maltose. Storage buffer was equilibration buffer with the addition of 0.27M sucrose and glycerol—PMSF and benzamidine were omitted.

Protein Purification from E. coli

Full length wild-type and kinase-inactive TcPINK1 was expressed in E. coli as maltose binding protein fusion (MBP) protein and purified as described previously (Woodroof et al, 2011). Briefly, BL21 Codon+ transformed cells were grown at 37° C. to an OD₆₀₀ of 0.3, then shifted to 16° C. and induced with 250 μM IPTG (isopropyl β-D-thiogalactoside) at OD₆₀₀ of 0.5. Cells were induced with 2500 IPTG at OD 0.6 and were further grown at 16° C. for 16 hrs. Cells were pelleted at 3000 rpm, and then lysed by sonication in lysis buffer. Lysates were clarified by centrifugation at 30,000 g for 30 min at 4° C. followed by incubation with 1 ml per litre of culture of amylose resin for 1.5 h at 4° C. The resin was washed thoroughly in wash buffer, then equilibration buffer, and proteins were then eluted. Proteins were dialysed overnight at 4° C. into storage buffer, snap frozen and stored at −80° C. until use.

MBP-ATP13A2 and MBP-PLA2G6 were purified by similar methods. GST-a-synuclein, GST-parkin, GST-DJ1, GST-LRRK2 kinase inactive (KI) (1326-end D2017A), GST-Omi, GST-GAK KI (D191A), GST-FBX07, untagged VPS35 (GST cleaved), GST-TRAP1, GST-PARL, GST-NCS1 and GST-Miro2 were purified by similar methods except that recombinant GST-fusion proteins were affinity purified on glutathione-Sepharose and eluted with buffer containing 20 mM glutathione. GST-VPS35 was cleaved with GST-PreScission protease at 4° C. overnight. His-UCHL1 was obtained from Ubiquigent (UK). Untagged parkin (His-SUMO cleaved) was expressed and purified by Helen Walden's laboratory (Chaugule et al, 2011).

### Antibodies

The antibody against PINK1 phospho-Thr²⁵⁷ (S114D) was generated by injection of the KLH (keyhole-limpet haemocyanin)-conjugated phospho-peptide CAGEYGAVpTYRKSKR (where pT is phospho-threonine) into sheep and was affinity-purified by positive and negative selection against the phospho- and de-phospho-peptides respectively. The antibody against parkin phospho-Ser⁶⁵ (S210D) was generated by injection of the KLH (keyhole-limpet haemocyanin)-conjugated phospho-peptide RDLDQQpSIVHIVQR (where pS is phospho-serine) into sheep and was affinity-purified by positive and negative selection against the phospho- and de-phospho-peptides respectively. The antibody against total Parkin (S966C) was raised against the recombinant GST-parkin full-length protein and successively affinity purified by positive and negative selection against recombinant fusion protein and GST respectively. Anti-human PINK1 rabbit polyclonal (aa 175-250) antibody was obtained from Novus Biologicals; anti-GAPDH mouse monoclonal from Millipore; anti-Parkin mouse monoclonal (Santa Cruz), anti-HSP60 rabbit polyclonal from Cell Signaling Technology. Anti-FLAG agarose beads were obtained from SIGMA.

### Immunoprecipitation and Immunoblotting

Immunoprecipitation of recombinant PINK1-FLAG was undertaken by standard methods with anti-FLAG agarose beads (Sigma); of HA-parkin with anti-HA agarose beads (Sigma); and of untagged parkin with anti-parkin antibody (S966C) covalently conjugated to protein G-Sepharose. Immunoprecipitates, as well as cell lysates in SDS sample buffer were subjected to SDS-PAGE and transferred to PVDF membranes. For immunoblotting, membranes were incubated for 60 mins with 1% TBST containing either 5% (wt/vol) skimmed milk powder (for total antibodies) or 5% (wt/vol) BSA (for phospho-specific antibodies). The antibodies were then incubated in the same buffer overnight at 4° C. with the indicated primary antibodies. Sheep total and phospho-specific antibodies were used at a concentration of 1 μg/ml, whereas commercial antibodies were diluted 1000-fold. The incubation with phospho-specific sheep antibodies was performed with the addition of 10 μg/ml of the dephosphopeptide antigen used to raise the antibody. Blots were washed with 0.1% TBST and incubated with secondary HRP-conjugated antibodies in 5% skimmed milk for 60 mins. After repeated washes, the signal was detected with the enhanced chemiluminescence and the X-ray films were processed in a Konica Minolta Medical SRX-101 film processor.

### Kinase Assays

In assays utilising E. coli expressed wild-type or kinase dead (D359A) MBP-TcPINK1, reactions were set up in a volume of 40 μl, with substrates at 1 μg and kinase at 0.5 μg in 50mMTris-HCl (pH 7.5), 0.1 mM EGTA, 10 mM MgCl2, 2 mM dithiothreitol (DTT) and 0.1 mM [γ-³²P] ATP (approximately 500 cpm/pmol). Assays were incubated at 30° C. with shaking at 1200 rpm and terminated after the indicated time by addition of SDS sample buffer. In mammalian HEK293 immunoprecipitation kinase assays, C-terminal-FLAG tagged wild-type or kinase dead (D384A) PINK1 was immunoprecipitated from 5 mg of mitochondrial enriched extracts using anti-FLAG agarose beads and activity measured in a reaction volume of 40 μl consisting of 50 mM Tris-HCl (pH 7.5), 0.1 mM EGTA, 10 mM MgCl2, 2 mM DTT, 0.1 mM [γ-³²P] ATP (2000cpm/pmol) and 5 μg of indicated substrate. Assays were incubated at 30° C. with shaking at 1200 rpm and terminated after 30 min by addition of SDS sample buffer. For all assays, reaction mixtures were resolved by SDS-PAGE. Proteins were detected by Coomassie staining and gels were imaged using an Epson scanner and dried completely using a gel dryer (Bio-Rad). Incorporation of [γ-32P] ATP into substrates was analysed by autoradiography using Amersham Hyper-Film ECL.

### Lambda Phosphatase Assay

C-terminal-FLAG tagged wild-type or kinase-inactive (D384A) PINK1 were immunoprecipitated from 5 mg of mitochondrial enriched extracts using anti-FLAG agarose beads. Wild-type PINK1 was incubated with or without 1000U of Lambda phosphatase (NEB) in a reaction volume of 40 μl consisting of 50 mM Tris pH 7.5, 1 mM MnCl₂ and 2 mM DTT. In addition wild-type PINK1 was treated with 1000U of lambda phosphatase in the presence of 50 mM EDTA. Assays were incubated at 30° C. for 30 min with shaking at 1200 rpm. The beads were washed three times in 50 mM Tris pH7.5, 0.1 mM EGTA and then utilized in an in vitro kinase assay with GST-parkin UBL (1-108) as the substrate. Samples were further analyzed as described above.

### Mapping the Site on Human Parkin Phosphorylated by TcPINK1

GST-Parkin (1 μg) purified from E. coli was incubated with 2 μg of either wild type MBP-TcPINK1 (1-570) or kinase dead MBP-TcPINK1 (D359A) for 60 mins at 30° C. in 50mMTris-HCl (pH 7.5), 0.1 mM EGTA, 10 mM MgCl2, 2 mM dithiothreitol (DTT) and 0.1 mM [γ-³²P] ATP (approximately 20,000 cpm/pmol) in a total reaction volume of 25 μl. The reaction was terminated by addition of LDS sample buffer with 10 mM DTT, boiled, and alkylated with 50 mM iodoacetamide before samples were subjected to electrophoresis on a Bis-Tris 4-12% polyacrylamide gel, which was then stained with Colloidal Coomassie blue (Invitrogen). Phosphorylated parkin was digested with trypsin and >95% of ³²P radioactivity incorporated in the gel bands was recovered. Peptides were chromatographed on a reverse phase HPLC Vydac C₁₅ column (Cat#218TP5215, Separations Group, Hesperia, Calif.) equilibrated in 0.1% (v/v) trifluoroacetic acid and the column developed with a linear acetonitrile gradient at a flow rate of 0.2 ml/min and fractions (0.1 ml each) were collected and analysed for ³²P radioactivity by Cerenkov counting. Isolated phosphopeptides were analysed by LC-MS-MS on a proxeon Easy-nLC nano liquid chromatography system coupled to a Thermo LTQ-orbitrap mass spectrometer. The resultant data files were searched using Mascot (www.matrixscience.com) run on an in-house system against a database containing the parkin sequence, with a 10 p.p.m. mass accuracy for precursor ions, a 0.8 Da tolerance for fragment ions, and allowing for Phospho (ST), Phospho (Y), Oxidation (M) and Dioxidation (M) as variable modifications. Individual MS/MS spectra were inspected using Xcalibur 2.2 software. The site of phosphorylation of these ³²P-labelled peptides was determined by solid-phase Edman degradation on an Applied Biosystems 494C sequencer of the peptide coupled to Sequelon-AA membrane (Applied Biosystems) as described previously (Campbell & Morrice, 2002).

### Large Scale Immunoprecipitation of Mitochondrial Human PINK1 Followed by Identification of Phosphorylated Thr²⁵⁷ by Mass Spectrometry

10 mg of mitochondrial extract from Flp-In T-Rex HEK 293 cell lines stably PINK1-FLAG were subjected to immunoprecipitation with anti-FLAG-agarose and then eluted in LDS sample buffer. Samples were boiled with 10 mM DTT, and then alkylated with 50 mM iodoacetamide before being subjected to electrophoresis on a Bis-Tris 4-12% gradient polyacrylamide gel, which was then stained with Colloidal Coomassie blue. Coomassie-stained bands migrating with the expected molecular mass of PINK1-FLAG were excised from the gel and digested with trypsin and samples were analysed either by an Applied Biosystems 4000 Q-TRAP system with precursor ion scanning as described previously (Williamson et al, 2006) or on the LTQ-Orbitrap Velos system with multistage activation.

### Large Scale Immunoprecipitation of Human Parkin Followed by Identification of Phosphorylated Ser⁶⁵ by Mass Spectrometry

Flp-In T-Rex HEK 293 cell lines stably expressing empty vector, wild-type or kinase-inactive PINK1-FLAG were sequentially co-transfected with HA-parkin, induced with 0.1 μg/ml of Doxycycline and then incubated with 100 CCCP or DMSO control for 3 hours before whole cell lysis. Approximately 30 mg of lysate was subjected to immunoprecipitation with anti-FLAG-agarose and then eluted in LDS sample buffer. Samples were boiled with 10 mM DTT, and then alkylated with 50 mM iodoacetamide before being subjected to electrophoresis on a Bis-Tris 10% polyacrylamide gel, which was then stained with Colloidal Coomassie blue. Coomassie-stained bands migrating with the expected molecular mass of parkin were excised from the gel and digested with trypsin and samples underwent phosphosite analysis with LTQ-Orbitrap Velos. Individual MS/MS spectra of phosphopeptides were inspected using Xcalibur 2.2 software.

### N-Terminal Edman Sequencing

HEK293 cells were transiently transfected with wild-type PINK1-FLAG and then underwent whole cell lysis. 100 mg of lysate was subjected to immunoprecipitation with anti-FLAG agarose and then eluted in LDS sample buffer. Samples were boiled with 10 mM DTT, and then alkylated with 50 mM iodoacetamide before being subjected to electrophoresis on a Bis-Tris 10% polyacrylamide gel, which was then transferred to Immobilon PVDF (Polyvinylidene difluoride) membrane and stained briefly with Cooomassie Blue. The band corresponding to the processed form of PINK1 was excised and subjected to Edman degradation in an Applied Biosystems ProCise 494 Sequencer. The resulting HPLC profiles were analysed with Model 610 software (Applied Biosystems).

### Results

Insect PINK1 Phosphorylates Parkin In Vitro.

As some of the known Parkinson's disease-linked proteins may function in a signalling network (Muqit & Alessi, 2009), we tested whether catalytically active recombinant insect TcPINK1 could directly phosphorylate 11 different Parkinson's disease-linked proteins and 7 putative PINK1 interacting proteins (FIGS. 1A & 1B). Strikingly, this revealed that wild type but not kinase-inactive TcPINK1 phosphorylated full-length parkin, but not any of the other proteins tested including Omi (Plun-Favreau et al, 2007), TRAP1 (Pridgeon et al, 2007) or Miro2 (Wang et al, 2011; Weihofen et al, 2009) (FIGS. 1A and 1B).

Insect PINK1 Phosphorylates Parkin at Ser⁶⁵, a Highly Conserved Residue within the Ubl Domain.

TcPINK1 phosphorylated parkin in a time-dependent manner reaching a maximal stoichiometry of phosphorylation of −0.25 moles of ³²P-phosphate per mole of protein (FIG. 1C). ³²P-labelled parkin was digested with trypsin and analyzed by chromatography on a C₁₅ column. Two major ³²P-labeled phosphopeptides were observed (FIG. 1D). A combination of solid-phase Edman sequencing and mass spectrometry revealed that both of these encompassed variants of a peptide phosphorylated at Ser⁶⁵ (FIGS. 5A & B). Ser⁶⁵ is located within the N-terminal Ubl domain of parkin and is highly conserved from mammals to invertebrates (FIG. 1E). Mutating Ser⁶⁵ to Ala prevented phosphorylation of full-length parkin or an N-terminal parkin fragment containing the isolated Ubl domain (aa 1-108) by TcPINK1 thereby confirming that this residue represents the major site of PINK1 phosphorylation (FIG. 1F). The isolated Ubl domain of parkin was phosphorylated to a significantly higher stoichiometry than full-length parkin in a parallel experiment (FIG. 1F).

Evidence that Human PINK1 Phosphorylates Parkin at Ser⁶⁵In Vivo.

To study whether parkin was phosphorylated by PINK1 in cells we over-expressed full-length parkin in HEK293 Flp-In TRex cells stably expressing wild-type PINK1, or kinase-inactive PINK1 (D384A) (FIG. 2A). Cells were treated with or without the mitochondrial uncoupling agent, CCCP, for 3 hours—conditions that induce stabilisation and activation of PINK1 at the mitochondria (see introduction and also see subsequent FIG. 3). Parkin was immunoprecipitated and phosphorylation site analysis undertaken by mass spectrometry. This strikingly revealed that parkin was phosphorylated at Ser⁶⁵, but only in cells expressing wild type human PINK1 that had been stimulated with CCCP (FIG. 2A). No detectable phosphorylation of Ser⁶⁵ was observed in the absence of CCCP treatment or in cells expressing kinase-inactive PINK1 (FIG. 2A). We also detected phosphorylation of a previously reported phosphorylation site (Ser¹³¹). In contrast to Ser⁶⁵, phosphorylation of Ser¹³¹ was constitutive and not modulated by CCCP or PINK1 (FIG. 2A). We failed to detect phosphorylation of parkin at another previously reported site (Thr¹⁷⁵) (Kim et al, 2008). We next raised a phospho-specific antibody that specifically recognised parkin phosphorylated at Ser⁶⁵ and used this to confirm that parkin phosphorylation at Ser⁶⁵ is induced by CCCP (3 hours treatment) in HEK293 cells expressing human wild-type PINK1 in vivo. Interestingly we also observed trace phosphorylation of parkin Ser⁶⁵ in cells not overexpressing PINK1 treated with CCCP suggesting that there may be endogenous PINK1 present in HEK293 cells which is also able to phosphorylate parkin (FIG. 2B and see also FIG. 3D).

Human PINK1 Isolated from CCCP Treated Cells is Capable of Phosphorylating Parkin.

Wild type or kinase-inactive PINK1 was immunoprecipitated from the mitochondrial fraction of cells treated with CCCP and tested to see whether it could phosphorylate the Ubl domain of parkin in vitro. This revealed that wild type but not kinase-inactive PINK1 isolated from CCCP stimulated cells phosphorylated the isolated Ubl domain of parkin in a manner that was prevented by mutation of Ser⁶⁵ to Ala (FIG. 2C). In contrast, wild type PINK1 isolated from non-CCCP treated cells, failed to phosphorylate the Ubl domain of parkin (FIG. 2C). These observations indicate that CCCP treatment is inducing the activation of human PINK1 thereby rendering it capable of phosphorylating parkin at Ser⁶⁵.

Evidence that CCCP Promotes PINK1 Autophosphorylation.

We next studied the localisation and electrophoretic mobility of wild-type and kinase-inactive human PINK1 in response to CCCP (see Materials and Methods). Similar to previous observations (Geisler et al, 2010; Matsuda et al, 2010; Narendra et al, 2010; Vives-Bauza et al, 2010) in non-CCCP treated cells, full-length as well as a truncated form of wild type and kinase-inactive PINK1 was present in cytoplasmic and mitochondrial fractions (FIG. 3A). N-terminal Edman sequencing of the truncated from of PINK1 confirmed that it commenced at residue 104 (FIG. 6) consistent with previous work indicating that human PINK1 is proteolysed between residues Ala103-Phe104 by the mitochondrial rhomboid protease, PARL (Deas et al, 2011; Jin et al, 2010; Meissner et al, 2011; Whitworth et al, 2008). A 3 hour CCCP treatment induced a marked increase in the levels of the full-length form of PINK1 associated with the mitochondria, which was accompanied by a large reduction in cytoplasmic levels of PINK1 (FIG. 3A). We also observed that CCCP led to a significant increase in levels of full length PINK1 in whole cell extracts (FIG. 3A) consistent with CCCP stabilising full-length PINK1. Levels of full-length kinase-inactive PINK1 were also stabilised following CCCP treatment (FIG. 3A).

We also noticed that CCCP treatment induced a significant decrease in the electrophoretic mobility (band-shift) of the wild type but not kinase-inactive PINK1 (FIG. 3A). This prompted us to investigate whether CCCP stimulated phosphorylation of any residues on PINK1. We undertook mass spectrometric phosphopeptide analysis of wild type and kinase-inactive full length PINK1 after immunoprecipitation from mitochondrial fractions of CCCP treated cells. This revealed that several residues of PINK1 were phosphorylated in CCCP-treated cells at low stoichiometry making the identification of phosphorylation sites challenging. Thus far we have only been able to identify one of these phosphorylation sites that corresponds to Thr²⁵⁷ (FIG. 3B and FIG. 7). Employing a phosphospecific Thr²⁵⁷antibody that we raised, we were able to confirm that CCCP treatment markedly stimulated phosphorylation of wild type but not kinase-inactive PINK1 at Thr²⁵⁷ (FIG. 3C), suggesting this residue is an autophosphorylation site. Mutation of Thr257 to Ala abolished detection of phosphorylated PINK1 confirming the specificity of the Thr²⁵⁷antibody (FIG. 3C). Parkin Ser⁶⁵ phosphorylation was still observed in CCCP-treated cells expressing the PINK1 Thr257A mutant suggesting that phosphorylation of this residue is not required for CCCP-induced PINK1 activation in vivo (FIG. 3D). We also observed that the Thr257A mutation did not prevent the CCCP-induced band-shift (FIGS. 3C and 3D). Thr²⁵⁷ is located within the second insert-region (residues 247-270) and like many autophosphorylation sites in other protein kinases, is not highly conserved between species. Nevertheless monitoring phosphorylation of this residue could serve as a useful marker for PINK1 activity.

We next investigated how phosphatase treatment affects CCCP-induced PINK1 activity. We found that lambda phosphatase treatment of PINK1 isolated from CCCP treated cells induced complete dephosphorylation of Thr²⁵⁷, and also resulted in a significant inhibition of PINK1 activity as judged by its ability to phosphorylate parkin. Addition of the lambda phosphatase inhibitor EDTA prevented dephosphorylation of Thr²⁵⁷ and loss of ability of PINK1 to phosphorylate parkin. This suggests that phosphorylation of PINK1 at additional sites other than Thr²⁵⁷ may be important in mediating the activation of PINK1 induced by CCCP (FIG. 3E). We also observed that phosphatase treatment did not collapse the CCCP-induced bandshift (FIG. 3E), indicating that either phosphatase resistant sites or another type of protein modification mediates the bandshift.

Time Course of PINK1 Activation, Autophosphorylation and Phosphorylation of Parkin.

We next investigated the time-course of the PINK1 stabilisation, band-shift, autophosphorylation of Thr²⁵⁷, and ability of PINK1 to phosphorylate parkin following CCCP treatment. This revealed that the stabilisation of full-length PINK1 at the mitochondria is rapid with significant stabilisation seen within 5 min of CCCP treatment and is maximal by 40 min and then sustained for up to 3 hours (FIG. 4A). Loss of the cleaved form of PINK1 observed in the cytosol is particularly rapid and almost disappears within 5 min of CCCP treatment (FIG. 4B). However, the appearance of the band-shift and autophosphorylation of Thr²⁵⁷ occurred more slowly and was observed only after 40 min of CCCP treatment and sustained for up to 3 h (FIG. 4A). There was no phosphorylation of Thr²⁵⁷ or bandshift of cytoplasmic associated PINK1 indicating that mitochondrial association is required for this (FIG. 4B). We studied the time-course of PINK1 activation by assessing the ability of immunoprecipitated mitochondrial PINK1 to phosphorylate parkin in vitro and found that PINK1 activation occurred around 40 mins of CCCP treatment and maximal at 3 hours (FIG. 4C). In contrast, monitoring parkin Ser⁶⁵ phosphorylation using the phospho-specific antibody against p-Ser⁶⁵ indicated that parkin Ser⁶⁵ phosphorylation occurs at 5 mins (FIG. 4D) and becomes maximal and sustained from 40 mins onwards. This suggests that the kinetics of PINK1 activation against its substrate is significantly faster than the kinetics of PINK1 autophosphorylation.

### DISCUSSION

Our observations provide strong evidence that PINK1 is activated following stabilisation of full length PINK1 at the mitochondrial membrane after CCCP treatment and suggest that PINK1 directly phosphorylates parkin at Ser⁶⁵. Observations indicating that Drosophila dPINK1 and dParkin null flies have similar degenerative phenotypes (Clark et al, 2006; Park et al, 2006) and that over-expression of parkin rescues the phenotype of dPINK1 null Drosophila (but not the converse) are consistent with PINK1 acting upstream of parkin (Clark et al, 2006; Park et al, 2006). The ability of PINK1 to regulate mitochondrial dynamics in mammalian cells as well as Drosophila has also been suggested to be dependent upon parkin (Cui et al, 2011; Whitworth & Pallanck, 2009; Yang et al, 2008; Yu et al, 2011). The finding that humans with loss of function mutations in either PINK1 or parkin display indistinguishable clinical presentation of Parkinson's disease, also argues in favour of a major connection between PINK1 and parkin in humans (Abeliovich & Flint Beal, 2006).

The Ser⁶⁵ PINK1 phosphorylation site on parkin is highly conserved as are the surrounding residues. This is what would be expected for a key PINK1 phosphorylation site on an effector protein. Recent work suggests that the Ubl domain of parkin acts as an auto-inhibitory domain by binding to the C-terminal region and preventing catalytic activity (Chaugule et al, 2011). Based on this it is tempting to speculate that phosphorylation of Ser⁶⁵ within the core of the Ubl domain would relieve the auto-inhibition thereby activating the E3 ligase activity of parkin. If parkin was regulated in this manner, then loss of function mutations in PINK1 would lead to suppression of parkin E3 ligase activity and result in reduced ubiquitylation of parkins targets. This would also account for why over-expression of parkin in dPINK1 null Drosophila restores ubiquitylation of targets and rescues the null phenotype. Previous work has also found that PINK1 promotes the translocation of parkin to the mitochondria and that catalytic activity of PINK1 may be required for this. It would therefore be interesting to investigate whether Ser⁶⁵ phosphorylation promotes recruitment of parkin to the mitochondria. It is possible that the key parkin targets are located at the mitochondria and several candidate mitochondrial substrates for parkin have been proposed including Mitofusin1 (Ziviani et al, 2010) and VDAC1 (Geisler et al, 2010). In future work it would be vital to test whether phosphorylation of parkin at Ser⁶⁵ influences its ability to ubiquitylate these or other targets and define how this links to Parkinson's disease.

In previous work, employing a positional scanning peptide library approach, we elaborated an artificial peptide substrate termed PINKtide, that had the sequence WIpYRRSPRRR, which was phosphorylated by an insect orthologue, TcPINK1, albeit weakly with a Vmax of 8 U/mg and a Km of 4930 (Woodroof et al., 2011). This contrasts with optimal peptides for other active protein kinases that can usually be phosphorylated with a Vmax of 100-1000 U/mg and Km of less than 10 μM. Mutation of the +1 proline in PINKtide to other residues tested inhibited phosphorylation, suggesting this residue might comprise a key determinant for PINK1 phosphorylation (Woodroof et al., 2011). However, the sequence encompassing Ser⁶⁵ of parkin, DLDQQSIVHI, is quite dissimilar from PINKtide and does not possess a+1 Pro residue. Our parkin data suggests that a+1 Pro residue is not an essential determinant for PINK1 phosphorylation. Previous studies, based on co-immunoprecipitation and co-localisation experiments (Xiong et al, 2009)(Sha et al, 2010) have reported that PINK1 and parkin bind and therefore it is possible that additional docking interactions between PINK1 and parkin enable Ser⁶⁵ to be efficiently phosphorylated. This may also explain why a short peptide encompassing Ser⁶⁵ synthesised by our lab was not significantly phosphorylated by TcPINK1 (data not shown). Inspection of various NMR structures (Safadi et al, 2011; Sakata et al, 2003; Tashiro et al, 2003) as well as the crystal structure of the isolated Ubl domain of mammalian parkin (Tomoo et al, 2008) reveal that Ser⁶⁵ lies within the fifth β-strand that makes up the ubiquitin-like fold. It is likely that phosphorylation of this residue would result in a significant conformational change of the Ubl domain. In future work it would be critical to study the biophysical interaction between PINK1 and parkin in more detail and establish how important this is in enabling PINK1 to phosphorylate Ser⁶⁵. It would also be interesting to determine the structure of the Ser⁶⁵ phosphorylated Ubl domain of parkin to ascertain how phosphorylation affects the conformation of this domain.

There has been one previous report that human PINK1 isolated from non-CCCP treated cells can directly phosphorylate parkin at a single threonine residue, Thr¹⁷⁵, (Kim et al, 2008). In that study a deletion fragment of PINK1 spanning residues 200-581 was utilised that would be predicted to be missing approximately the first 50 amino acids of the N-lobe of the PINK1 kinase domain including the conserved glycine rich motif (residues 163-169), which in other kinases is essential for coordinating ATP. This construct of PINK1 would not be expected to be active. Moreover, in that report the kinase-inactive mutant of PINK1[200-581] fragment still exhibited substantial kinase activity towards parkin (Kim et al, 2008). Taken together these findings indicate that the phosphorylation of Thr¹⁷⁵ observed in this study was likely to be mediated by a contaminating kinase. Our experiments have also identified Ser¹³¹ as a phosphorylation site in parkin that is constitutively phosphorylated and not influenced by PINK1 or CCCP (FIG. 2A). Ser¹³¹lies within the linker region of parkin between the Ubl and RINGO domain and unlike Ser⁶⁵ is not fully conserved in lower organisms (e.g. leucine in Drosophila). A previous in vitro study has suggested that Ser¹³¹ may be phosphorylated by cdk5 (Avraham et al, 2007), and further work would be required to define the importance of this phosphorylation site.

Our findings suggest that the full-length form of PINK1 becomes rapidly stabilised within 5 minutes of CCCP treatment and this also coincides with the disappearance of the cleaved form of PINK1 (FIG. 4B). PINK1 also becomes activated at 5 mins and reaches maximal activity at 40 mins as assessed by monitoring phosphorylation of parkin Ser⁶⁵ within cells (FIG. 4D). However, the time course of PINK1 autophosphorylation at Thr²⁵⁷ takes longer requiring around 40 min and then activation is sustained for at least up to 3 h (FIGS. 4A and 4C). It is not uncommon for kinases to exhibit differential kinetics of catalytic activity for autophosphorylation as compared to substrate phosphorylation. The kinetics for kinase activity against a substrate generally occurs faster than autophosphorylation and is regarded as a more reliable read-out of kinase activation. The kinetics of PINK1 activation as judged by the ability of immunoprecipitated PINK1 to phosphorylate parkin in vitro occurred later than the cell-based read-out of parkin Ser⁶⁵ phosphorylation (FIG. 4C). In our hands the in vitro activity of human PINK1 is low and this assay may not be sensitive enough to detect PINK1 activation at earlier time-points. What drives the stabilisation of full length PINK1 at the mitochondria and destabilisation of cleaved PINK1 is unknown at present. One proposal is that the rhomboid PARL protease is rapidly inhibited following CCCP-induced depolarisation in mitochondria thereby leading to stabilisation of the full-length form of PINK1 (Meissner et al, 2011). The striking disappearance of the cleaved form of PINK1 in the cytoplasm within 5 min also suggests that CCCP could trigger rapid degradation of this form of PINK1. In future work it would be important not only to investigate how full length PINK1 is stabilised and the cleaved form of PINK1 destabilised, but also to determine whether simple recruitment of PINK1 to mitochondria is sufficient to induce its activation or whether additional depolarisation and/or stabilisation of the full length form of PINK1 is a pre-requisite for subsequent activation of PINK1. It would also be essential to discover the mechanism by which PINK1 is activated at the mitochondria following CCCP treatment. Our data suggests that activation of PINK1 can be observed after immunoprecipitation and extensive washing of the immunoprecipitate, which may be consistent with a covalent modification. Lambda phosphatase treatment substantially reduced PINK1 activation suggesting that phosphorylation is required for full activation (FIG. 3E). In future work it would be essential to establish what are the key phosphorylation and/or other covalent modifications induced by CCCP and whether these are responsible for triggering activation of PINK1. It is also possible that PINK1 becomes associated with a non-covalent activator at the mitochondrial membrane such as another protein or a small molecule second messenger. It would be interesting to investigate whether CCCP mediated generation of an intermediate molecular species such as reactive oxygen species (ROS), could modify PINK1 leading to its activation.

Our data suggests that PINK1, like many kinases, autophosphorylate at Thr²⁵⁷ and probably other residues after it is activated. This is also associated with an electrophoretic mobility shift on a polyacrylamide gel after CCCP treatment of wild-type but not kinase-inactive PINK1, which would be incapable of autophosphorylating (FIG. 3A). It should be noted that this bandshift was best observed by resolving proteins on an 8% isocratic polyacrylamide gel and was much less pronounced on gradient gels (FIG. 7). This may explain why the bandshift of wild type PINK1 following CCCP treatment has not been reported before. Although our data suggests that autophosphorylation of Thr²⁵⁷ is not critical for triggering the activation of PINK1 we still feel that phospho-antibodies that recognise Thr²⁵⁷ are likely to be useful reporter of PINK1 activation.

Given the large body of evidence implicating mitochondrial dysfunction in Parkinson's disease (Abou-Sleiman et al, 2006), it would be important to explore further in subsequent work whether Ser⁶⁵ and optionally Thr²⁵⁷ phosphorylation could have utility as specific biomarkers for Parkinson's disease progression. It would also be interesting to look at Ser⁶⁵ and Thr²⁵⁷ phosphorylation in transgenic mouse models of Parkinson's disease (e.g. a-synuclein) to determine whether this pathway is implicated in other genetic forms of Parkinson's disease. It would also be important to examine Ser⁶⁵ and Thr²⁵⁷ phosphorylation in brains and cell lines of patients with parkin and PINK1 mutations and more importantly sporadic Parkinson's disease.

In conclusion, we have added important new information that supports the notion that PINK1 and parkin function in a common signalling pathway. Our data suggest that CCCP induces stabilisation and activation of PINK1 at the mitochondria enabling it to directly phosphorylate parkin at Ser⁶⁵ within the N-terminal Ubl domain. We also provide evidence that once activated, PINK1 autophosphorylates at several residues and this is associated with an electrophoretic bandshift on a polyacrylamide gel. We have identified one of these autophosphorylated residues as Thr²⁵⁷ and provided evidence that this could serve as a reporter for PINK1 activation. Our findings provide reagents and a framework for exciting follow-up studies. Firstly it will be crucial to understand how CCCP and potentially other agents that target the mitochondria activate PINK1. It will also be essential to understand how phosphorylation of parkin at Ser⁶⁵ influences its function and to identify the key substrates that parkin ubiquitylates. Hopefully such information could provide valuable clues as to how disruption of the PINK1-parkin signalling pathway leads to Parkinson's disease and whether this pathway is also disrupted in patients with the sporadic form of the disease. These studies are imperative as they could lead to new ideas for therapies to treat and monitor Parkinson's disease in the future.

### REFERENCES

Abeliovich A, Flint Beal M (2006) Parkinsonism genes: culprits and clues. J Neurochem 99: 1062-1072
Abou-Sleiman P M, Muqit M M, Wood N W (2006) Expanding insights of mitochondrial dysfunction in Parkinson's disease. Nat Rev Neurosci 7: 207-219
Avraham E, Rott R, Liani E, Szargel R, Engelender S (2007) Phosphorylation of Parkin by the cyclin-dependent kinase 5 at the linker region modulates its ubiquitin-ligase activity and aggregation. The Journal of biological chemistry 282: 12842-12850
Campbell D G, Morrice N A (2002) Identification of protein phosphorylation sites by a combination of mass spectrometry and solid phase Edman sequencing. Journal of biomolecular techniques: JBT 13: 119-130
Chaugule V K, Burchell L, Barber K R, Sidhu A, Leslie S J, Shaw G S, Walden H (2011) Autoregulation of Parkin activity through its ubiquitin-like domain. The EMBO journal 30: 2853-2867
Clark I E, Dodson M W, Jiang C, Cao J H, Huh J R, Seol J H, Yoo S J, Hay B A, Guo M (2006) Drosophila pink1 is required for mitochondrial function and interacts genetically with parkin. Nature 441: 1162-1166
Cui M, Tang X, Christian W V, Yoon Y, Tieu K (2011) Perturbations in mitochondrial dynamics induced by human mutant PINK1 can be rescued by the mitochondrial division inhibitor mdivi-1. J Biol Chem 285: 11740-11752
Deas E, Plun-Favreau H, Gandhi S, Desmond H, Kjaer S, Loh S H, Renton A E, Harvey R J, Whitworth A J, Martins L M, Abramov A Y, Wood N W (2011) PINK1 cleavage at position A103 by the mitochondrial protease PARL. Hum Mol Genet 20: 867-879
Geisler S, Holmstrom K M, Skujat D, Fiesel F C, Rothfuss O C, Kahle P J, Springer W (2010) PINK1/Parkin-mediated mitophagy is dependent on VDAC1 and p62/SQSTM1. Nat Cell Biol 12: 119-131
Jin S M, Lazarou M, Wang C, Kane L A, Narendra D P, Youle R J (2010) Mitochondrial membrane potential regulates PINK1 import and proteolytic destabilization by PARL. J Cell Biol 191: 933-942
Kim Y, Park J, Kim S, Song S, Kwon S K, Lee S H, Kitada T, Kim J M, Chung J (2008) PINK1 controls mitochondrial localization of Parkin through direct phosphorylation. Biochemical and biophysical research communications 377: 975-980
Kitada T, Asakawa S, Hattori N, Matsumine H, Yamamura Y, Minoshima S, Yokochi M, Mizuno Y, Shimizu N (1998) Mutations in the parkin gene cause autosomal recessive juvenile parkinsonism. Nature 392: 605-608
Matsuda N, Sato S, Shiba K, Okatsu K, Saisho K, Gautier C A, Sou Y S, Saiki S, Kawajiri S, Sato F, Kimura M, Komatsu M, Hattori N, Tanaka K (2010) PINK1 stabilized by mitochondrial depolarization recruits Parkin to damaged mitochondria and activates latent Parkin for mitophagy. J Cell Biol 189: 211-221
Meissner C, Lorenz H, Weihofen A, Selkoe D J, Lemberg M K (2011) The mitochondrial intramembrane protease PARL cleaves human Pink1 to regulate Pink1 trafficking. J Neurochem 117: 856-867
Muqit M M, Abou-Sleiman P M, Saurin A T, Harvey K, Gandhi S, Deas E, Eaton S, Payne Smith M D, Venner K, Matilla A, Healy D G, Gilks W P, Lees A J, Holton J, Revesz T, Parker P J, Harvey R J, Wood N W, Latchman D S (2006) Altered cleavage and localization of PINK1 to aggresomes in the presence of proteasomal stress. J Neurochem 98: 156-169
Muqit M M, Alessi D R (2009) A meeting of minds: overcoming roadblocks in the development of therapies for neurodegenerative disorders. EMBO Mol Med 1: 139-141
Narendra D P, Jin S M, Tanaka A, Suen D F, Gautier C A, Shen J, Cookson M R, Youle R J (2010) PINK1 is selectively stabilized on impaired mitochondria to activate Parkin. PLoS Biol 8: e1000298
Park J, Lee S B, Lee S, Kim Y, Song S, Kim S, Bae E, Kim J, Shong M, Kim J M, Chung J (2006) Mitochondrial dysfunction in Drosophila PINK1 mutants is complemented by parkin. Nature 441: 1157-1161
Plun-Favreau H, Klupsch K, Moisoi N, Gandhi S, Kjaer S, Frith D, Harvey K, Deas E, Harvey R J, McDonald N, Wood N W, Martins L M, Downward J (2007) The mitochondrial protease HtrA2 is regulated by Parkinson's disease-associated kinase PINK1. Nat Cell Biol 9: 1243-1252
Pridgeon J W, Olzmann J A, Chin L S, Li L (2007) PINK1 protects against oxidative stress by phosphorylating mitochondrial chaperone TRAP1. PLoS Biol 5: e172
Reed S E, Staley E M, Mayginnes J P, Pintel D J, Tullis G E (2006) Transfection of mammalian cells using linear polyethylenimine is a simple and effective means of producing recombinant adeno-associated virus vectors. Journal of virological methods 138: 85-98
Safadi S S, Barber K R, Shaw G S (2011) Impact of autosomal recessive juvenile Parkinson's disease mutations on the structure and interactions of the parkin ubiquitin-like domain. Biochemistry 50: 2603-2610
Sakata E, Yamaguchi Y, Kurimoto E, Kikuchi J, Yokoyama S, Yamada S, Kawahara H, Yokosawa H, Hattori N, Mizuno Y, Tanaka K, Kato K (2003) Parkin binds the Rpn10 subunit of 26S proteasomes through its ubiquitin-like domain. EMBO reports 4: 301-306
Sha D, Chin L S, Li L (2010) Phosphorylation of parkin by Parkinson disease-linked kinase PINK1 activates parkin E3 ligase function and NF-kappaB signaling. Hum Mol Genet 19: 352-363
Takatori S, Ito G, Iwatsubo T (2008) Cytoplasmic localization and proteasomal degradation of N-terminally cleaved form of PINK1. Neurosci Lett 430: 13-17
Tashiro M, Okubo S, Shimotakahara S, Hatanaka H, Yasuda H, Kainosho M, Yokoyama S, Shindo H (2003) NMR structure of ubiquitin-like domain in PARKIN: gene product of familial Parkinson's disease. Journal of biomolecular NMR 25: 153-156
Tomoo K, Mukai Y, In Y, Miyagawa H, Kitamura K, Yamano A, Shindo H, Ishida T (2008) Crystal structure and molecular dynamics simulation of ubiquitin-like domain of murine parkin. Biochim Biophys Acta 1784: 1059-1067
Valente E M, Abou-Sleiman P M, Caputo V, Muqit M M, Harvey K, Gispert S, Ali Z, Del Turco D, Bentivoglio A R, Healy D G, Albanese A, Nussbaum R, Gonzalez-Maldonado R, Deller T, Salvi S, Cortelli P, Gilks W P, Latchman D S, Harvey R J, Dallapiccola B, Auburger G, Wood N W (2004) Hereditary early-onset Parkinson's disease caused by mutations in PINK1. Science 304: 1158-1160
Vives-Bauza C, Zhou C, Huang Y, Cui M, de Vries R L, Kim J, May J, Tocilescu M A, Liu W, Ko H S, Magrane J, Moore D J, Dawson V L, Grailhe R, Dawson T M, Li C, Tieu K, Przedborski S (2010) PINK1-dependent recruitment of Parkin to mitochondria in mitophagy. Proceedings of the National Academy of Sciences of the United States of America 107: 378-383
Wang X, Winter D, Ashrafi G, Schlehe J, Wong Y L, Selkoe D, Rice S, Steen J, LaVoie M J, Schwarz T L (2011) PINK1 and Parkin target Miro for phosphorylation and degradation to arrest mitochondrial motility. Cell 147: 893-906
Weihofen A, Thomas K J, Ostaszewski B L, Cookson M R, Selkoe D J (2009) Pink1 forms a multiprotein complex with Miro and Milton, linking Pink1 function to mitochondrial trafficking. Biochemistry 48: 2045-2052
Whitworth A J, Lee J R, Ho V M, Flick R, Chowdhury R, McQuibban G A (2008) Rhomboid-7 and HtrA2/Omi act in a common pathway with the Parkinson's disease factors Pink1 and Parkin. Dis Model Mech 1: 168-174; discussion 173
Whitworth A J, Pallanck L J (2009) The PINK1/Parkin pathway: a mitochondrial quality control system? J Bioenerg Biomembr 41: 499-503
Williamson B L, Marchese J, Morrice N A (2006) Automated identification and quantification of protein phosphorylation sites by L C/M S on a hybrid triple quadrupole linear ion trap mass spectrometer. Molecular & cellular proteomics: MCP 5: 337-346
Woodroof H I, Pogson J H, Begley M, Cantley L C, Deak M, Campbell D G, van Aalten DMF, Whitworth A J, Alessi D R, Muqit M M (2011) Discovery of catalytically active orthologues of the Parkinson's disease kinase PINK1: analysis of substrate specificity and impact of mutations. Open Biology 1: rsob. 110012
Xiong H, Wang D, Chen L, Choo Y S, Ma H, Tang C, Xia K, Jiang W, Ronai Z, Zhuang X, Zhang Z (2009) Parkin, PINK1, and D J-1 form a ubiquitin E3 ligase complex promoting unfolded protein degradation. J Clin Invest 119: 650-660
Yang Y, Ouyang Y, Yang L, Beal M F, McQuibban A, Vogel H, Lu B (2008) Pink1 regulates mitochondrial dynamics through interaction with the fission/fusion machinery. Proceedings of the National Academy of Sciences of the United States of America 105: 7070-7075
Yu W, Sun Y, Guo S, Lu B (2011) The PINK1/Parkin pathway regulates mitochondrial dynamics and function in mammalian hippocampal and dopaminergic neurons. Hum Mol Genet 20: 3227-3240
Ziviani E, Tao R N, Whitworth A J (2010) Drosophila parkin requires PINK1 for mitochondrial translocation and ubiquitinates mitofusin. Proceedings of the National Academy of Sciences of the United States of America 107: 5018-5023

1-10. (canceled)

11. An in vitro method of diagnosing Parkinson's disease, monitoring of progression of Parkinson's disease, assessing response to therapy and/or screening drugs for treating Parkinson's disease, the method comprising detecting phosphorylation of amino acid residues of the parkin and PINK1 protein.

12. The method according to claim 11 comprising identification of phosphorylation of the Ser⁶⁵ residue of parkin (numbering according to GenBank: BAA25751.1).

13. The method according to claim 12 further comprising identifying phosphorylation of Thr²⁵⁷ of PINK1.

14. The method according to claim 11 conducted on a biological sample, from the subject.

15. The method according to claim 14 wherein the biological sample is a sample of CSF.

16. The method according to claim 11 wherein detection of said phosphorylated residue(s) is conducted using a Pro-Q® Diamond phosphorylation gel stain, a phosphor specific antibody, mass spectrometry, chromatographic 2-D gel separation, competitive inhibition assay, and/or a binding assay.

17. The method according to claim 11 further comprising detecting other signs, symptoms, conducting clinical tests of Parkinson's disease, and/or other Parkinson's disease biomarkers.

18. The method according to claim 11 wherein the method is conducted on at least two different types of fluid and/or tissue.

19. A molecule that specifically binds to phosphorylated or unphosphorylated Ser⁶⁵ of parkin or Thr²⁵⁷ of PINK1 or region comprising said residue(s).

20. The molecule according to claim 19 which is an antibody.

21. A method according to claim 11 wherein the detection of phosphorylation of amino acids residues of the parkin and PINK1 proteins is carried out using antibodies specific for phosphorylated residues on parkin and PINK1.

22. The method according to claim 21 wherein the antibodies for use in detecting phosphorylated residues of parkin and PINK1 comprise antibodies which specifically bind phosphorylated Ser⁶⁵ of parkin and Thr²⁵⁷ of PINK1.

23. A kit for diagnosing Parkinson's disease, monitoring progression of the disease and/or assessing response to therapy, the kit comprising at least one marker specific reagent that specifically binds to phosphorylated or unphosphorylated Ser⁶⁵ of parkin or Thr²⁵⁷ of PINK1 or region comprising said residue(s).

24. The kit according to claim 23 wherein the kit comprises antibodies which specifically bind phosphorylated Ser⁶⁵ of parkin and Thr²⁵⁷ of PINK1.

25. The kit according to claim 23 further comprising a container for a sample collected from a subject.


SEQUENCE LISTING (8 sequences)

SEQ ID NO: 1 - protein, 26 aa, Homo sapiens
RNDWTVQNCDLDQQSIVHIVQRPWRK

SEQ ID NO: 2 - protein, 15 aa, Homo sapiens
  MOD_RES (9)..(9): PHOSPHORYLATION
CAGEYGAVTYRKSKR

SEQ ID NO: 3 - protein, 14 aa, Homo sapiens
  MOD_RES (7)..(7): PHOSPHORYLATION
RDLDQQSIVHIVQR

SEQ ID NO: 4 - protein, 10 aa, Artificial
  artificial peptide
WIYRRSPRRR

SEQ ID NO: 5 - protein, 10 aa, Homo sapiens
DLDQQSIVHI

SEQ ID NO: 6 - protein, 13 aa, Homo sapiens
  MOD_RES (11)..(11): PHOSPHORYLATION
VALAGEYGAVTYR

SEQ ID NO: 7 - protein, 14 aa, Homo sapiens
  MOD_RES (11)..(11): PHOSPHORYLATION
VALAGEYGAVTYRK

SEQ ID NO: 8 - protein, 14 aa, Homo sapiens
  MISC_FEATURE (2)..(2): X is A or I
RXVFLAFGXGLGLI